(19) United States
(12) Patent Application Publication
(10) Pub. No.: US 2012/0252066 A1
(43) Pub. Date: Oct. 4, 2012

(54) METHODS OF FOAM CONTROL

(76) Inventors: Meng H. Heng, Belmont, CA (US); Michael Bodo, Cupertino, CA (US)

(21) Appl. No.: 13/433,036

(22) Filed: Mar. 28, 2012

Related U.S. Application Data
(60) Provisional application No. 61/469,067, filed on Mar. 29, 2011.

(51) Int. Cl. C12P 21/00; C12P 19/44

(52) U.S. Cl. 435/69.1; 435/74

(57) ABSTRACT

The invention relates to a method for decreasing foam formation as well as maximizing expression of a biosurfactant in a microorganism. The methods encompasses precipitating a biosurfactant from the microorganism which results in decreased form formation.

### RELATED APPLICATIONS AND INCORPORATION BY REFERENCE

This application claims priority to U.S. provisional patent application Ser. No. 61/469,067 filed Mar. 29, 2011. Reference is made to international patent application Ser. No. PCT/US2009/046783 filed 9 Jun. 2009, which published as PCT Publication No. WO 2009/152176 on 17 Dec. 2009 and Serial No. PCT/US2010/044964 filed 10 Aug. 2010, which published as PCT Publication No. WO 2011/019686 on 17 Feb. 2011.

The foregoing applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The invention relates to a method for controlling foaming of a biosurfactant that foams during production thereof by a host cell in a fermentation medium when the host cell extracellularly secretes the biosurfactant and the biosurfactant is soluble in the fermentation medium. The method comprises or consists essentially of, contemporaneously with production of the biosurfactant by the host cell, insolubilizing the biosurfactant. In this manner, foaming is controlled as the insolubilized biosurfactant does not foam. By this method, the foam reduction index is greater than 1, and/or the foam reduction index is greater than 2, and/or the foam reduction index is greater than 3. Likewise, additionally or alternatively by this method, the concentration of soluble biosurfactant in the fermentation media is at most about 1 g/kg. Additionally or alternatively; and/or at least 25% of the biosurfactant produced is insolubilized. Additionally or alternatively, the method is performed without addition of antifoam; or provides the ability to reduce the amount of antifoam that would be used without insolubilizing the biosurfactant, such as a 25% or 30% or 40% 50% or 60% or 65% or 70% or 75% or 80% or 85% or 90% or 95% or greater reduction in amount of antifoam that would be used without insolubilizing the biosurfactant. Also additionally or alternatively, while the invention can be performed in a batch or fed-batch manner, the invention advantageously relates to such methods that are continuous. The invention also advantageously relates to such methods wherein the biosurfactant is a hydrophobin, such as hydrophobin II. The invention also advantageously relates to such methods wherein the biosurfactant is a glycolipid such as rhamnolipid and sophorolipid, or a lipopeptide such as surfactin. Even further the invention relates to apparatus for performing the methods of the invention, especially continuous methods of the invention. Further still, the invention relates to methods of the invention wherein the insolubilizing of biosurfactant is induced by adding a precipitation agent, such as a salt, alcohol, water miscible organic solvent, water soluble polymer or a cationic polymer, or by changing pH or by changing temperature.

### BACKGROUND OF THE INVENTION

Surfactants are widely used chemicals for various industries, and are mainly synthesized chemically. Surfactants produced by a variety of microorganisms are gaining attention due to their unique properties such as higher bio-degradability and lower toxicity profiles than the synthetic counterparts. However, the availability and cost of such biologically produced surfactants are limited due, in part, to lack of efficient production methods.

An efficient system for industrial scale protein or enzyme production is by aerobic submerged fermentation followed by aqueous based recovery steps to isolate the product(s) of interest. However, foam control is critical to achieve the efficiency.

Foaming is a serious problem in the chemical industry, especially for biochemical processes. Foam is often produced as an unwanted consequence in the manufacture of various substances such as surfactants and proteins, particularly in processes involving significant shear forces near air-liquid interfaces, such as those involving aeration, pumping or agitation. Aerobic submerged fermentation relies on adequate aeration to supply oxygen required by the microorganisms to grow and produce product of interest. The introduction of air into the fermentation broth to provide oxygen required by the microorganism generates foam. The presence of foam during fermentation generally has negative impacts on its performance, including reduction of fermentor working volume or productivity, and a risk of contamination associated with a “foam out”, such as the production of a foam column or foam head above the liquid fermentation broth of sufficient height that it exits the fermentation vessel through venting or pipes.

Additives such as antifoam or defoamers are commonly used to mitigate foam formation during fermentation. Antifoam agents, as necessary, are added during the recovery steps to control foam. Some recovery processes are negatively impacted by the presence of antifoam, especially membrane-based separation processes. Depending on the end-use application of the proteins or enzymes, the antifoam agents employed during its production process may or may not need to be removed.

However, chemical methods of foam control are not always desired with respect to the problems (i.e. contamination, reduction of mass transfer) they may cause, especially in the food, feed and pharmaceutical industries, where product quality is of great importance. Because antifoam agents are usually hydrophobic, they are difficult to sterilize, which may pose issues in the food and pharmaceutical industries. In addition, regulatory requirements in these industries limit the chemistries that are acceptable for use in antifoams and defoamers.

Unfortunately, conventional submerged aerobic fermentation and recovery processes for industrial scale protein or enzyme production cannot be efficiently applied to the production of biosurfactants, i.e., biologically produced surfactant molecules. The surfactancy of these molecules will, under the same culturing conditions, give rise to much more foam in the fermentation broth, than would the same microorganism not expressing the biosurfactant molecule.

Addition of antifoam agents is not usually a satisfactory solution to the problem. Not only are copious amounts of antifoam agents necessary to prevent excessive foam formation, but removal of the antifoam agents is generally required for the surfactant to function as intended in the target applications. In some cases, even addition of copious amount of antifoam agents and operating at relatively low working percentage of fermentor volume is not effective in controlling the foaming. The challenge associated with excessive foaming and uncontrolled foaming by use of antifoam agents continues in the downstream recovery steps. Because surfactants are sought for their detergency, the antifoam agents added during the production step must generally be removed.

Citation or identification of any document in this application is not an admission that such document is available as prior art to the present invention.

### SUMMARY OF THE INVENTION

The invention is based, in part, on Applicants' surprising discovery that addition of a precipitation agent to a fermentation broth results in precipitating the biologically-expressed surfactant as well as a reduction in foaming, wherein the foam does not return.

This invention describes methods and/or uses relating to the control of foam in the production of an aqueous solution which may comprise one or more surfactants expressed by a microorganism. This may be accomplished by appropriate conditioning of the solutions such that the foam forming surfactants are made insoluble. The appropriate conditioning may include precipitation, crystallization, and/or any other manipulation that renders the surfactant insoluble or reduces the critical micelle concentration.

The invention encompasses a method and/or a use for controlling foaming of biosurfactant that foams during production thereof by a host cell in a fermentation medium when the host cell extracellularly secretes the foaming biosurfactant and the biosurfactant is soluble in the fermentation medium, which may comprise, contemporaneously with production of the biosurfactant by the host cell, insolubilizing the biosurfactant, whereby foaming is controlled as the insolubilized biosurfactant does not foam.

The invention also encompasses a method and/or a use for controlling foaming of biosurfactant that foams during production thereof by a host cell in a fermentation medium when the host cell extracellularly secretes the biosurfactant and the biosurfactant is soluble in the fermentation medium, which may comprise, contemporaneously with production of the biosurfactant by the host cell, insolubilizing the biosurfactant, whereby foaming is controlled as the insolubilized biosurfactant does not foam, wherein the foam reduction index is greater than 1, and/or the foam reduction index is greater than 2, and/or the foam reduction index is greater than 3; and/or the concentration of soluble biosurfactant in the fermentation media is at most about 1 g/kg; and/or at least 25% of biosurfactant produced is insolubilized; and/or the method is performed without addition of antifoam; and/or the method is performed with a reduced amount of antifoam in comparison with the method run without insolubilizing the biosurfactant.

The invention provides a method and/or a use for reducing or eliminating the foam formation caused by the biosurfactant when it is in solution, by reducing the soluble concentration of biosurfactant through appropriate choice of process conditions. Process conditions that result in reduced solubility of the biosurfactant depend on the nature of the biosurfactant. Such process conditions can encompass the proper choice of physical conditions such as temperature and/or pressure. Such process conditions can furthermore encompass the chemical composition of the liquid medium in which the biosurfactant is present. The possible choices of such compositions are numerous and well known to those skilled in the art of bioprocessing. Chemical approaches to modulate solubility conditions encompass use of additives that render the biosurfactant insoluble, including pH buffer chemicals, salts of mineral or organic acids or bases, alcohols, organic solvents, polymers, polyols, proteins, adsorbents, nucleic acids, lipids, This list of solubility modifying chemicals is not intended to be exclusive or limiting.

The invention also comprehends a method and/or a use of preparing a biosurfactant comprising foam control or aspect(s) thereof herein provided.

The invention accordingly relates to the in situ insolubilization or contemporaneous with expression, in situ insolubilization of surfactant(s) expressed e.g by a microorganism or biosurfactant, including batch process(es) or continuous process(es) for preparing a biosurfactant comprising in situ insolubilization or contemporaneous with expression, in situ insolubilization of the biosurfactant. The insolubilization may be by precipitation, crystallization, [, because of EP1320595 Yoneda et al.; Syldatk et al./1984; Desai et al./1993] and/or any other manipulation that renders the surfactant insoluble or reduces the critical micelle concentration. Advantageously, the insolubilization comprises or consists essentially of adding a precipitation agent, such as a salt, alcohol, water miscible organic solvent, water soluble polymer or a cationic polymer (such as, but not limited to, C581), or the insolubilization comprises or consists essentially of pH adjustment, such as decreasing pH. The insolubilization can comprise or consist of adjusting temperature and/or pressure, e.g., increasing temperature or heating. In particularly advantageous embodiments, the use of an antifoam in preparing the biosurfactant is decreased or avoided altogether. In advantageous embodiments, the biosurfactant, e.g., hydrophobin such as hydrophobin II, is present in solution in a concentration of less than about 0.1 g/kg.

The present invention also relates to a method and/or a use for controlling foaming of biosurfactant in a solution that foams during production which may comprise contemporaneously during the production of the biosurfactant at points where conditions can give rise to foam formation, insolubilizing the biosurfactant, whereby foaming is controlled as the insolubilized biosurfactant does not foam.

In another embodiment, the invention also pertains to a method and/or a use for controlling foaming of biosurfactant that foams during production which may comprise contemporaneously with production of the biosurfactant in a solution by the host cell, insolubilizing the biosurfactant, controlling foaming such that: the foam reduction index is greater than 1, and/or the foam reduction index is greater than 2, and/or the foam reduction index is greater than 3; and/or the concentration of soluble biosurfactant in the solution is at most about 1 g/kg; and/or at least 25% of, biosurfactant produced is insolubilized; and/or the method is performed without addition of antifoam; and/or the method is performed with a reduced amount of antifoam in comparison with the method run without insolubilizing.

In yet another embodiment, the invention relates to a method and/or a use for controlling foaming of biosurfactant during production which may comprise controlling conditions of a composition during production of the biosurfactant to reduce foam, which may comprise adjusting conditions in the composition to reduce foaming such that the foam reduction index is greater than 1, and/or the foam reduction index is greater than 2, and/or the foam reduction index is greater than 3; the concentration of soluble biosurfactant in the fermentation media is at most about 1 g/kg; and/or at least 25% of biosurfactant produced is insolubilized; and/or the method is performed without addition of antifoam; and/or the method is performed with a reduced amount of antifoam in comparison with the method run without insolubilizing; and/or the method is performed at a pH of about 4.0.

The benefits of this invention apply to all stages of biosurfactant processing, including fermentation, recovery, formulation, storage, handling, and transportation. In particular, the benefits especially apply at a stage of biosurfactant processing involving aeration, such as but not limited to, mixing, pumping and release of gas.

The invention further comprehends an apparatus as herein described, including employed in the practice of method(s) or process(es) or aspect(s) thereof as herein described.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Mention is especially made of the use of “consisting essentially of” and “consists essentially of” to distinguish over, to any extent available as art, US Patent Publication No. 20100151525 and any document equivalent thereto, e.g., by way of subject matter and/or patent law (e.g., by being or claiming priority from or being in the same family as EP08171868). For example, in the instant invention, any use of carrageenan need not be accompanied by decreasing the pH, particularly, for example, below 3.0 or 3.5 and/or adjusting ionic strength; and, any decreasing of pH need not be accompanied by use of carrageenan and/or adjusting ionic strength, and any adjusting of ionic strength need not be accompanied by decreasing pH and/or carrageenan use. Hence, “consists essentially of” and “consists essentially of” excludes elements of the prior art, such as adding carrageenan and having pH below 3.5, or 3, or adding carrageenan, having pH below 3.5 or 3 and adjusting ionic strength.

Mention is also made that certain terms are particularly also meant to exclude that which is in any document that may be art. For example, the term ‘biosurfactant’ is particularly meant to exclude the enzyme subject matter of PCT Publication No. WO 2009/152176. Similarly, expressions of practicing without or in the absence of an added antifoam agent are to distinguish over documents that allow for the presence or the addition of antifoam agent(s), e.g., US Patent Publication No. 20100291630 and any document equivalent thereto.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 depicts a hydrophobin solution after mixing (left) and a hydrophobin solution after mixing, and heat treated (right).

FIG. 2 depicts the MALDI-TOF spectra of the hydrophobin produced using the modified fermentation. The peak at 7180 corresponds to the full length hydrophobin molecule.

FIGS. 3A and 3B depict a representative bioreactor. Cells, media, and/or nutrients may be provided to reactor 100 via inputs 102. Input 102 may include valve 104 used to control the delivery of organisms and/or media to the vessel. Cells and media may be provided via input 102. Multiple sensors 106 may be positioned at locations throughout reactor 100. Sensor 106 provide data to controllers 108, 110. Controllers 108, 110 are capable of controlling an amount of cells, media, nutrients, precipitating agent and/or other components. The precipitated component may detected using sensors 106. In some embodiments a window 116 may be present in reactor 100 to allow a user to observe conditions in the reactor. Controller 108 is connected to output valve 112. Controller 110 may direct valve 112 to open to allow precipitate to leave the tank via output 114. In some embodiments, user input may allow control to direct valve 112 to open and/or close as needed. Nutrients may be provided to reactor using input 118. Input 118 may be coupled to delivery device 120 to provide nutrients to reactor 100. Some embodiments include mixer 122 to promote mixing of the components in the reactor.

FIG. 4 shows the reduction in foam formation in a Bacillus licheniformis fermentation broth containing surfactin measured following calcium chloride treatment as described in Example 20.

### DETAILED DESCRIPTION OF THE INVENTION

Unless defined otherwise, all technical and scientific terms used herein have the same meaning as commonly understood by one of ordinary skill in the art. The following abbreviations and/or terms are defined for clarity.

As used herein, a “biosurfactant” or a “biologically produced surfactant” pertains to a substance that causes foaming. A biosurfactant or biologically produced surfactant may decrease surface tension, such as the interfacial tension between water and a hydrophobic liquid, or between water and air, and that may be produced or obtained from a biological system. A biosurfactant or biologically produced surfactant may be a protein, a glycolipid, a lipopeptide, a lipoprotein, a phospholipid, a neutral lipid or a fatty acid. Biosurfactants include hydrophobins. Biosurfactants include lipopeptides and lipoproteins such as surfactin, peptide-lipid, serrawettin, viscosin, subtilisin, gramicidins, polymyxins. Biosurfactants include glycolipids such as rhamnolipids, sophorolipids, trehalolipids and cellobiolipids. Biosurfactants include polymers such as emulsan, biodispersan, mannan-lipid-protein, liposan, carbohydrate-protein-lipid, protein PA. Biosurfactants include particulates such as vesicles, fimbriae, and whole cells. Biosurfactants include glycosides such as saponins. Biosurfactants include fibrous proteins such as fibroin. The biosurfactant may occur naturally or it may be a mutagenized or genetically engineered variant not found in nature. This includes biosurfactant variants that have been engineered for lower solubility to help control foaming by lowering the biosurfactant solubility according to this invention. Biosurfactants include, but are not limited to, related biosurfactants, derivative biosurfactants, variant biosurfactants and homologous biosurfactants as described herein.

As used herein, a “biological system” comprises or is derived from a living organism such as a microbe, a plant, a fungus, an insect, a vertebrate or a life form created by synthetic biology. The living organism can be a variant not found in nature that is obtained by classical breeding, clone selection, mutagenesis and similar methods to create genetic diversity, or it can be a genetically engineered organism obtained by recombinant DNA technology. The living organism can be used in its entirety or it can be the source of components such as organ culture, plant cultivars, suspension cell cultures, adhering cell cultures or cell free preparations.

The biological system may or may not contain living cells when it sequesters the biosurfactant. The biological system may be found and collected from natural sources, it may be farmed, cultivated or it may be grown under industrial conditions. The biological system may synthesize the biosurfactant from precursors or nutrients supplied or it may enrich the biosurfactant from its environment.

As used herein, “production” relates to manufacturing methods for the production of chemicals and biological products, which includes, but is not limited to, harvest, collection, compaction, exsanguination, maceration, homogenization, mashing, brewing, fermentation, recovery, solid liquid separation, cell separation, centrifugation, filtration (such as vacuum filtration), formulation, storage or transportation.

As used herein, “process conditions” refer to a solvent and/or a choice of physical parameters (such as, but not limited to, temperature, pressure, mixing or pH) involved in the methods of the present invention.

As used herein, a “solvent” or “solution” relates to a liquid that may contain suspended particles other than an insoluble biosurfactant, such as, but not limited to, body parts, plant fragments, living or dead cells [because of EP1320595 Yoneda et al.; Syldatk et al./1984; Desai et al./1993].

As used herein, “soluble” relates to a substance which is dissolved in a solvent or solution.

As used herein, “foam” relates to a substance that is formed by trapping gaseous bubbles in a liquid, in a gel or in a semisolid.

As used herein, “overrun” is a calculated value which relates to the volume of a foamed solution minus the starting volume, divided by the starting volume, reported as a fraction or percentage. An overrun of zero means the solution contains no foam. A number close to zero means the solution has very low foam. In cases where an initial sample already contains foam, initial weight replaces initial volume in the calculation.

As used herein, “foam reduction index” or “foam control index” or “foam knockout index” is a measure of the effectiveness of a treatment for controlling the foam. It is the ratio of the overrun of an untreated solution to a treated solution. A foam reduction index equal to about 1 means untreated and treated biosurfactant solution have the same overrun, in other words, the treatment gives no improvement. Any number greater than 1 means there is foam reduction, the treatment gives improvement.

As used herein, “foam control”, “foam reduction” or “foam knockout” relates to actions that reduce foam in a solution by preventing or discouraging or destroying or destructing foam

As used herein, the terms “polypeptide” and “protein” are used interchangeably to refer to polymers of any length comprising amino acid residues linked by peptide bonds. The conventional one-letter or three-letter code for amino acid residues is used herein. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, D-amino acids, etc.), as well as other modifications known in the art.

As used herein, a “culture solution” is a liquid comprising a biosurfactant of interest and other soluble or insoluble components. Such components include other proteins, non-proteinaceous impurities such as cells or cell debris, nucleic acids, polysaccharides, lipids, chemicals such as antifoam, flocculants, salts, sugars, vitamins, growth factors, precipitants, and the like. A “culture solution” may also be referred to as “protein solution,” “liquid media,” “diafiltered broth,” “clarified broth,” “concentrate,” “conditioned medium,” “fermentation broth,” “lysed broth,” “lysate,” “cell broth,” or simply “broth.” The cells, if present, may be bacterial, fungal, plant, animal, human, insect, synthetic, etc.

As used herein, the term “recovery” refers to a process in which a liquid culture comprising a biosurfactant and one or more undesirable components is subjected to processes to separate the biosurfactant from at least some of the undesirable components, such as water, cells and cell debris, other proteins, amino acids, polysaccharides, sugars, polyols, inorganic or organic salts, acids and bases, and particulate materials.

As used herein, a “biosurfactant product” refers to a biosurfactant preparation suitable for providing to an end user, such as a customer. Biosurfactant products may include cells, cell debris, medium components, formulation excipients such as buffers, salts, preservative, reducing agents, sugars, polyols, surfactants, and the like, that are added or retained in order to prolong the functional shelf-life or facilitate the end use application of the biosurfactant.

As used herein, functionally and/or structurally similar biosurfactants are considered to be “related biosurfactants.” Such biosurfactants may be derived from organisms of different genera and/or species, or even different classes of organisms (e.g., bacteria and fungus). Related biosurfactants also encompass homologs determined by primary sequence analysis, determined by tertiary structure analysis, or determined by immunological cross-reactivity.

As used herein, the term “derivative biosurfactant” may refer to a protein-based biosurfactant which is derived from a biosurfactant by addition of one or more amino acids to either or both the N- and C-terminal end(s), substitution of one or more amino acids at one or a number of different sites in the amino acid sequence, and/or deletion of one or more amino acids at either or both ends of the protein or at one or more sites in the amino acid sequence, and/or insertion of one or more amino acids at one or more sites in the amino acid sequence. The preparation of a biosurfactant derivative may be achieved by modifying a DNA sequence which encodes for the native protein, transformation of that DNA sequence into a suitable host, and expression of the modified DNA sequence to form the derivative protein. A “derivative biosurfactant” may also encompass biosurfactant derivatives where either lipid or carbohydrate moieties have been attached to protein backbone either during or after synthesis.

As used herein, the term “derivative biosurfactant” or “variant biosurfactant” may refer to a lipid and/or sugar based biosurfactant which is derived from a biosurfactant by addition of one or more lipids and/or sugars, substitution of one or more lipids and/or sugars at one or a number of different sites, and/or deletion of one or more lipids and/or sugars at either or both ends of the molecule or at one or more sites within the structure, and/or insertion of one or more lipids and/or sugars at one or more sites in the structure.

Related (and derivative) biosurfactants include “variant biosurfactant.” A variant protein-based biosurfactant differs from a reference/parent biosurfactant, e.g., a wild-type biosurfactant, by substitutions, deletions, and/or insertions at small number of amino acid residues. The number of differing amino acid residues may be one or more, for example, 1, 2, 3, 4, 5, 6, 7, 8, 9, 10, 15, 20, 30, 40, 50, or more amino acid residues. Variant biosurfactants share at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99%, or more, amino acid sequence identity with a wildtype biosurfactant. A variant biosurfactant may also differ from a reference biosurfactant in selected motifs, domains, epitopes, conserved regions, and the like.

As used herein, the term “analogous sequence” refers to a sequence within a protein-based biosurfactant that provides similar function, tertiary structure, and/or conserved residues as the biosurfactant. For example, in epitope regions that contain an alpha-helix or a beta-sheet structure, the replacement amino acids in the analogous sequence preferably maintain the same specific structure. The term also refers to nucleotide sequences, as well as amino acid sequences. In some embodiments, analogous sequences are developed such that the replacement amino acids result in a variant enzyme showing a similar or improved function. In some embodiments, the tertiary structure and/or conserved residues of the amino acids in the biosurfactant are located at or near the segment or fragment of interest. Thus, where the segment or fragment of interest contains, for example, an alpha-helix or a beta-sheet structure, the replacement amino acids preferably maintain that specific structure.

As used herein, the term “homologous biosurfactant” refers to a biosurfactant that has similar activity and/or structure to a reference biosurfactant. It is not intended that homologs necessarily be evolutionarily related. Thus, it is intended that the term encompass the same, similar, or corresponding biosurfactant(s) (i.e., in terms of structure and function) obtained from different organisms. In some embodiments, it is desirable to identify a homolog that has a quaternary, tertiary and/or primary structure similar to the reference biosurfactant.

The degree of homology between sequences may be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, Wis.); and Devereux et al. (1984) Nucleic Acids Res. 12:387-395).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-360). The method is similar to that described by Higgins and Sharp (Higgins and Sharp (1989) CABIOS 5:151-153). Useful PILEUP parameters including a default gap weight of 3.00, a default gap length weight of 0.10, and weighted end gaps. Another example of a useful algorithm is the BLAST algorithm, described by Altschul et al. (Altschul et al. (1990) J. Mol. Biol. 215:403-410; and Karlin et al. (1993) Proc. Natl. Acad. Sci. USA 90:5873-5787). One particularly useful BLAST program is the WU-BLAST-2 program (See, Altschul et al. (1996) Meth. Enzymol. 266:460-480). Parameters “W,” “T,” and “X” determine the sensitivity and speed of the alignment. The BLAST program uses as defaults a word-length (W) of 11, the BLOSUM62 scoring matrix (See, Henikoff and Henikoff (1989) Proc. Natl. Acad. Sci. USA 89:10915) alignments (B) of 50, expectation (E) of 10, M′5, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Sequence identity may be determined using known programs such as BLAST, ALIGN, and CLUSTAL using standard parameters. (See e.g., Altschul, et al. (1990) J. Mol. Biol. 215:403-410; Henikoff et al. (1989) Proc. Natl. Acad. Sci. USA 89:10915; Karin et al. (1993) Proc. Natl. Acad. Sci. USA 90:5873; and Higgins et al. (1988) Gene 73:237-244). Software for performing BLAST analyses is publicly available through the National Center for Biotechnology Information. Also, databases may be searched using FASTA (Pearson et al. (1988) Proc. Natl. Acad. Sci. USA 85:2444-2448). One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, “wild-type” and “native” biosurfactants are those found in nature. The terms “wild-type sequence,” and “wild-type gene” are used interchangeably herein, to refer to a sequence that is native or naturally occurring in a host cell. In some embodiments, the wild-type sequence refers to a sequence of interest that is the starting point of a protein engineering project. The genes encoding the naturally-occurring protein may be obtained in accord with the general methods known to those skilled in the art. The methods generally comprise synthesizing labeled probes having putative sequences encoding regions of the biosurfactant, preparing genomic libraries from organisms expressing the protein, and screening the libraries for the gene of interest by hybridization to the probes. Positively hybridizing clones are then mapped and sequenced.

As used herein, “insoluble” or “insolubilized” pertains to poorly or very poorly soluble compounds. The insoluble fraction of a compound can be separated from the soluble fraction by high speed centrifugation of a 1 ml sample at 14,000×g for 10 minutes. Alternatively, the insoluble fraction can be separated from the soluble fraction by filtration through a 0.45 μm membrane filter such as for example a Millipore Durapore 1 L bottle top filter. The insoluble fraction would be in the pellet after centrifugation or remain on the filter after filtration. Alternatively, insolubilization of a previously clear solution can be detected by the appearance of turbidity or cloudiness. Alternatively, insoluble particles such as crystals or precipitates can be detected by light microscopy.

As used herein, “precipitation” pertains to the formation of a insoluble form of a compound from a solution of that compound caused by a chemical reaction or by a change in physical conditions. As used herein, a “precipitation agent” or “precipitant” pertains to an agent causing precipitation.

As used herein, “CMC” pertains to critical micelle concentration which may refer to the concentration of surfactants above which micelles form and almost all additional surfactants added to the system go to micelles. The CMC is an important characteristic of a surfactant. Before reaching the CMC, the surface tension changes strongly with the concentration of the surfactant. After reaching the CMC, the surface tension remains relatively constant or changes with a lower slope. The value of the CMC for a given dispersant in a given medium depends on temperature, pressure, and (sometimes strongly) on the presence and concentration of other surface active substances and electrolytes. Micelles only form above a critical micelle temperature. As used herein, lowering the CMC has the same effect as lowering the solubility of the biosurfactant in that it reduces the concentration of surfactant in solution and thus reduces foam formation.

As used herein, a “host cell” may be any cell in which the biosurfactant is produced, either naturally or by recombinant method. A host cell may include, but is not limited to, Agaricus spp. (e.g., Agaricus bisporus), an Agrocybe spp. (e.g., Agrocybe aegerita), an Ajellomyces spp., (e.g., Ajellomyces capsulatus, Ajellomyces dermatitidis), an Aspergillus spp. (e.g., Aspergillus arvii, Aspergillus brevipes, Aspergillus clavatus, Aspergillus duricaulis, Aspergillus ellipticus, Aspergillus flavus, Aspergillus fumigatus, Aspergillus fumisynnematus, Aspergillus lentulus, Aspergillus niger, Aspergillus oryzae, Aspergillus unilateralis, Aspergillus viridinutans), a Bacillus spp. (e.g., Bacillus licheniformis or Bacillus subtilis), a Beauveria spp. (e.g., Beauveria bassiana), a Candida spp. (e.g., Candida bogoriensis, Candida bombicola), a Claviceps spp. (e.g., Claviceps fusiformis), a Coccidioides spp., (e.g., Coccidioides posadasii), a Cochliobolus spp. (e.g., Cochliobolus heterostrophus), a Crinipellis spp. (e.g., Crinipellis perniciosa), a Cryphonectria spp. (e.g., Cryphonectria parasitica), a Davidiella spp. (e.g., Davidiella tassiana), a Dictyonema spp. (e.g., Dictyonema glabratum), an Emericella spp. (e.g., Emericella nidulans), an Escherichia spp. (e.g., Escherichia coli), a Flammulina spp. (e.g., Flammulina velutipes), a Fusarium spp. (e.g., Fusarium culmorum), a Gibberella spp. (e.g., Gibberella moniliformis), a Glomerella spp. (e.g., Glomerella graminicola), a Grifola spp. (e.g., Grifola frondosa), a Hansenula spp. (e.g., Hansenula polymorpha), a Heterobasidion spp. (e.g., Heterobasidion annosum), a Hypocrea spp. (e.g., Hypocrea jecorina, Hypocrea lixii, Hypocrea virens), a Kluyveromyces spp. (e.g., Kluyveromyces lactis), a Laccaria spp. (e.g., Laccaria bicolor), a Lentinula spp. (e.g., Lentinula edodes), a Magnaporthe spp. (e.g., Magnaporthe oryzae), a Marasmius spp. (e.g., Marasmius cladophyllus), a Moniliophthora spp. (e.g., Moniliophthora perniciosa), a Neosartorya spp. (e.g., Neosartorya aureola, Neosartorya fennelliae, Neosartorya fischeri, Neosartorya glabra, Neosartorya hiratsukae, Neosartorya nishimurae, Neosartorya otanii, Neosartorya pseudofischeri, Neosartorya quadricincta, Neosartorya spathulata, Neosartorya spinosa, Neosartorya stramenia, Neosartorya udagawae), a Neurospora spp. (e.g., Neurospora crassa, Neurospora discreta, Neurospora intermedia, Neurospora sitophila, Neurospora tetrasperma), a Ophiostoma spp. (e.g., Ophiostoma novo-ulmi, Ophiostoma quercus), a Paracoccidioides spp. (e.g., Paracoccidioides brasiliensis), a Passalora spp. (e.g., Passalora fulva), Paxillus filamentosusPaxillus involutus), a Penicillium spp. (e.g., Penicillium camemberti, Penicillium chrysogenum, Penicillium marneffei), a Phlebiopsis spp. (e.g., Phlebiopsis gigantea), a Pichia spp. (e.g., Pichia pastoris) a Pisolithus (e.g., Pisolithus tinctorius), a Pleurotus spp., (e.g., Pleurotus ostreatus), a Podospora spp. (e.g., Podospora anserina), a Postia spp. (e.g., Postia placenta), a Pseudomonas spp. (e.g., Pseudomonas aeruginosam, Pseudomonas fluorescens, Pseudomonas pyocyanea), a Pyrenophora spp. (e.g., Pyrenophora tritici-repentis), a Saccharomyces spp. (e.g., Saccharomyces cerevisiae), a Schizosaccharomyces spp. (e.g., Schizosaccharomyces pombe) a Schizophyllum spp. (e.g., Schizophyllum commune), a Streptomyces spp. (e.g., Streptomyces lividans), a Talaromyces spp. (e.g., Talaromyces stipitatus), a Torulopsis spp., a Trichoderma spp. (e.g., Trichoderma asperellum, Trichoderma atroviride, Trichoderma viride, Trichoderma reesii [formerly Hypocrea jecorina]), a Tricholoma spp. (e.g., Tricholoma terreum), a Uncinocarpus spp. (e.g., Uncinocarpus reesii), a Verticillium spp. (e.g., Verticillium dahliae), a Xanthodactylon spp. (e.g., Xanthodactylon flammeum), a Xanthoria spp. (e.g., Xanthoria calcicola, Xanthoria capensis, Xanthoria ectaneoides, Xanthoria flammea, Xanthoria karrooensis, Xanthoria ligulata, Xanthoria parietina, Xanthoria turbinata) or a Yarrowia spp. (e.g., Yarrowia lipolytica).

The methods of the present invention can be applied to the isolation of any biosurfactant from a culture solution. Advantageously, the biosurfactant is a soluble extracellular biosurfactant that is secreted by microorganisms. A group of exemplary biosurfactants are the hydrophobins, a class of cysteine-rich polypeptides expressed by and/or derived from filamentous fungi. Hydrophobins are small (˜100 amino acids) polypeptides known for their ability to form a hydrophobic coating on the surface of objects, including cells and man-made materials. First discovered in Schizophyllum commune in 1991, hydrophobins have now been recognized in a number of filamentous fungi. Based on differences in hydropathy and other biophysical properties, hydrophobins are categorized as being class I or class II. Hydrophobins are divided into two different classes (I or II) based on the characteristic spacing of conserved cystine residues and hydrophobicity patterns (Kershaw and Talbot 1998, Fungal Genet Biol 23:18-23 and Wösten 2001, Annu Rev Microbiol 55:625-646). See, e.g., Linder et al. (2005) FEMS Microbiology reviews, 29: 877-96 and Kubicek et al. (2008) BMC Evolutionary Biology, 8:4 for examples of class II hydrophobins.

The expression of hydrophobin conventionally requires the addition of a large amount of one or more antifoaming agents (i.e., antifoam) during fermentation. Otherwise, the foam produced by hydrophobin polypeptides saturates breather filters, contaminates vents, causes pressure build-up, and reduces protein yield. As a result, crude concentrates of hydrophobin conventionally contain residual amounts of antifoam, as well as host cell contaminants, which are undesirable in a hydrophobin preparation, particularly when the hydrophobin is intended as a food additive.

Hydrophobin can reversibly exist in forms having an apparent molecular weight that is greater than its actual molecular weight, which make hydrophobin well suited for recovery using the present methods. Liquid or foam containing hydrophobin can be continuously or periodically harvested from a fermentor for protein recovery as described, or harvested in batch at the end of a fermentation operation.

The hydrophobin can be any class I or class II hydrophobin known in the art, for example, hydrophobin from an Agaricus spp. (e.g., Agaricus bisporus), an Agrocybe spp. (e.g., Agrocybe aegerita), an Ajellomyces spp., (e.g., Ajellomyces capsulatus, Ajellomyces dermatitidis), an Aspergillus spp. (e.g., Aspergillus arvii, Aspergillus brevipes, Aspergillus clavatus, Aspergillus duricaulis, Aspergillus ellipticus, Aspergillus flavus, Aspergillus fumigatus, Aspergillus fumisynnematus, Aspergillus lentulus, Aspergillus niger, Aspergillus unilateralis, Aspergillus viridinutans), a Beauveria spp. (e.g., Beauveria bassiana), a Claviceps spp. (e.g., Claviceps fusiformis), a Coccidioides spp., (e.g., Coccidioides posadasii), a Cochliobolus spp. (e.g., Cochliobolus heterostrophus), a Crinipellis spp. (e.g., Crinipellis perniciosa), a Cryphonectria spp. (e.g., Cryphonectria parasitica), a Davidiella spp. (e.g., Davidiella tassiana), a Dictyonema spp. (e.g., Dictyonema glabratum), an Emericella spp. (e.g., Emericella nidulans), a Flammulina spp. (e.g., Flammulina velutipes), a Fusarium spp. (e.g., Fusarium culmorum), a Gibberella spp. (e.g., Gibberella moniliformis), a Glomerella spp. (e.g., Glomerella graminicola), a Grifola spp. (e.g., Grifola frondosa), a Heterobasidion spp. (e.g., Heterobasidion annosum), a Hypocrea spp. (e.g., Hypocrea jecorina, Hypocrea lixii, Hypocrea virens), a Laccaria spp. (e.g., Laccaria bicolor), a Lentinula spp. (e.g., Lentinula edodes), a Magnaporthe spp. (e.g., Magnaporthe oryzae), a Marasmius spp. (e.g., Marasmius cladophyllus), a Moniliophthora spp. (e.g., Moniliophthora perniciosa), a Neosartorya spp. (e.g., Neosartorya aureola, Neosartorya fennelliae, Neosartorya fischeri, Neosartorya glabra, Neosartorya hiratsukae, Neosartorya nishimurae, Neosartorya otanii, Neosartorya pseudofischeri, Neosartorya quadricincta, Neosartorya spathulata, Neosartorya spinosa, Neosartorya stramenia, Neosartorya udagawae), a Neurospora spp. (e.g., Neurospora crassa, Neurospora discreta, Neurospora intermedia, Neurospora sitophila, Neurospora tetrasperma), a Ophiostoma spp: (e.g., Ophiostoma novo-ulmi, Ophiostoma quercus), a Paracoccidioides spp. (e.g., Paracoccidioides brasiliensis), a Passalora spp. (e.g., Passalora fulva), Paxillus filamentosusPaxillus involutus), a Penicillium spp. (e.g., Penicillium camemberti, Penicillium chrysogenum, Penicillium marneffei), a Phlebiopsis spp. (e.g., Phlebiopsis gigantea), a Pisolithus (e.g., Pisolithus tinctorius), a Pleurotus spp., (e.g., Pleurotus ostreatus), a Podospora spp. (e.g., Podospora anserina), a Postia spp. (e.g., Postia placenta), a Pyrenophora spp. (e.g., Pyrenophora tritici-repentis), a Schizophyllum spp. (e.g., Schizophyllum commune), a Talaromyces spp. (e.g., Talaromyces stipitatus), a Trichoderma spp. (e.g., Trichoderma asperellum, Trichoderma atroviride, Trichoderma viride, Trichoderma reesii [formerly Hypocrea jecorina]), a Tricholoma spp. (e.g., Tricholoma terreum), a Uncinocarpus spp. (e.g., Uncinocarpus reesii), a Verticillium spp. (e.g., Verticillium dahliae), a Xanthodactylon spp. (e.g., Xanthodactylon flammeum), a Xanthoria spp. (e.g., Xanthoria calcicola, Xanthoria capensis, Xanthoria ectaneoides, Xanthoria flammea, Xanthoria karrooensis, Xanthoria ligulata, Xanthoria parietina, Xanthoria turbinata), and the like. Hydrophobins are reviewed in, e.g., Sunde, M et al. (2008) Micron 39:773-84; Linder, M. et al. (2005) FEMS Microbiol Rev. 29:877-96; and Wösten, H. et al. (2001) Ann. Rev. Microbiol. 55:625-46.

In a particularly advantageous embodiment, the hydrophobin is from a Trichoderma spp. (e.g., Trichoderma asperellum, Trichoderma atroviride, Trichoderma viride, Trichoderma reesii [formerly Hypocrea jecorina]), advantageously Trichoderma reseei.

Both class I and class II hydrophobins have been identified in fungi as secreted proteins that self-assemble at hydrophobilic interfaces into amphipathic films. Assemblages of class I hydrophobins are generally relatively insoluble whereas those of class II hydrophobins readily dissolve in a variety of solvents. Advantageously, hydrophobin is soluble in water, by which is meant that it is at least 0.1% soluble in water, preferably at least 0.5%. By at least 0.1% soluble is meant that no hydrophobin precipitates when 0.1 g of hydrophobin in 99.9 mL of water is subjected to 30,000 g centrifugation for 30 minutes at 20° C.

Applicants have observed that hydrophobin II produced by other methods can result in one or more amino acids clipped at the C terminus. From the methods of the present invention, in particular, if hydrophobin is precipitated or rendered insoluble, no clipping is observed.

Hydrophobin-like proteins (e.g.“chaplins”) have also been identified in filamentous bacteria, such as Actinomycete and Streptomyces sp. (WO01/74864; Talbot, 2003, Curr. Biol, 13: R696—R698). These bacterial proteins by contrast to fungal hydrophobins, may form only up to one disulphide bridge since they may have only two cysteine residues. Such proteins are an example of functional equivalents to hydrophobins, and another type of molecule within the ambit of biosurfactants of methods herein.

Rhamnolipids are a class of glycolipid produced by and/or derived from Pseudomonas aeruginosa, frequently cited as the best characterised of the bacterial surfactants. There are two main classes of rhamnolipids, mono-rhamnolipids and di-rhamnolipids; consisting of one or two rhamnose groups respectively. Rhamnolipids have been used broadly in the cosmetic industry for products such as moisturisers, toothpaste, condom lubricant and shampoo and are efficacious in bioremediation of organic and heavy metal polluted sites. They also facilitate degradation of waste hydrocarbons such as crude oil and vegetable oil by Pseudomonas aeruginosa.

Sophorolipids are found and excreted into the culture medium by Candida or related yeast species and are known as surfactants. The nature of the hydroxy fatty acid is characteristic, with the hydroxyl group being located on the n or n-1 carbon atom; the carbon chain length of 16, 17 or 18 is subject to modification by the composition of the growth medium. Sophorosides with unsaturated C18 fatty acids have been recognized in Candida bogoriensis. An unique sophorolipid was isolated from Torulopsis spp which differed from those already mentioned in that it was a macrocyclic lactone in which the carboxy group of the hydroxy fatty acid was esterified with the 4′ hydroxyl group of the terminal glucose in sophorose. Two acetate groups are also present in that lipid. Sophorolipids exhibit surfactant activity because of their amphiphilic structure. Among the sophorolipid producers, Candida bombicola is the most studied species because it produces sophorolipid species in large quantities. Sophorolipids have been shown to be useful in hard surface cleaning and automatic dishwashing rinse aid formulations.

Surfactin is a bacterial cyclic lipopeptide which is a very powerful surfactant Commonly used as an antibiotic. It is one of the 24 types of antibiotics produced by the Gram-positive endospore-forming bacteria Bacillus subtilis. Surfactin's structure consists of a peptide loop of seven amino acids (L-asparagine, L-leucine, glutamic acid, L-leucine, L-valine and two D-leucines), and a hydrophobic fatty acid chain thirteen to fifteen carbons long which allows its ability to penetrate cellular membranes. Surfactin, like other surfactants, affects the surface tension of liquids in which it is dissolved. It can lower the water's surface tension from 72 mN/m to 27 mN/m at a concentration as low as 20 μM.

Biosurfactants as described in U.S. Pat. Nos. 7,906,315; 7,893,015; 7,887,906; 7,858,334; 7,749,203; 7,581,594; 7,556,654; 7,541,321; 7,540,926; 7,473,363; 7,413,643; 7,325,603; 7,226,897; 7,198,680; 6,956,122; 6,921,390; 6,727,223; 6,582,730; 6,475,968; 6,389,820; 6,369,014; 6,346,281; 6,319,898; 6,262,038; 6,063,602; 6,060,287; 6,051,552; 5,866,376; 5,767,090; 5,635,392; 5,551,987; 5,417,879; 5,128,262; 4,943,390 and 4,640,767; and U.S. Patent Publication Nos. 20110065167; 20110027844; 20100323928; 20100168405; 20100144643; 20100143316; 20100004472; 20100000795; 20090288825; 20090269833; 20090203565; 20090170700; 20090148881; 20090098028; 20080296222; 20080293570; 20080193730; 20080085251; 20080023044; 20080023030; 20080020947; 20070249035; 20070249034; 20070215347; 20070134288; 20060106120; 20050271698; 20050266036; 20050227338; 20050176117; 20050106702; 20040251197; 20040244969; 20040231982; 20040156816; 20040152613; 20040022775; 20030096988; 20030018306; 20020176895; 20020123077 and 20020120101 may also be produced by the methods of the invention; see also Surfactant Science Series Volume 48, BIOSURFACTANTS, Production Properties Applications, Naim Kosaric, editor, CRC Press 1993.

Fermentation to produce the biosurfactant is carried out by culturing the host cell or microorganism in a liquid fermentation medium within a bioreactor or fermenter. The composition of the medium (e.g. nutrients, carbon source etc.), temperature and pH are chosen to provide appropriate conditions for growth of the culture and/or production of the biosurfactant. Air or oxygen-enriched air is normally sparged into the medium to provide oxygen for respiration of the culture.

The invention relates to adding any agent or treatment that causes a biosurfactant to precipitate to a culture solution that renders a biosurfactant insoluble. In particular, any agent or treatment that causes a biosurfactant to precipitate may be employed by the methods of the invention. Agents that cause a biosurfactant to precipitate include, but are not limited to, a salt, a polymer, an acid, a solvent or alcohol. Physical conditions that cause a biosurfactant to precipitate include, but are not limited to, a change in heat or a change in pH. The skilled artisan will understand that conditions to cause a biosurfactant to precipitate may include a precipitation agent, a change in a physical condition or a combination of both.

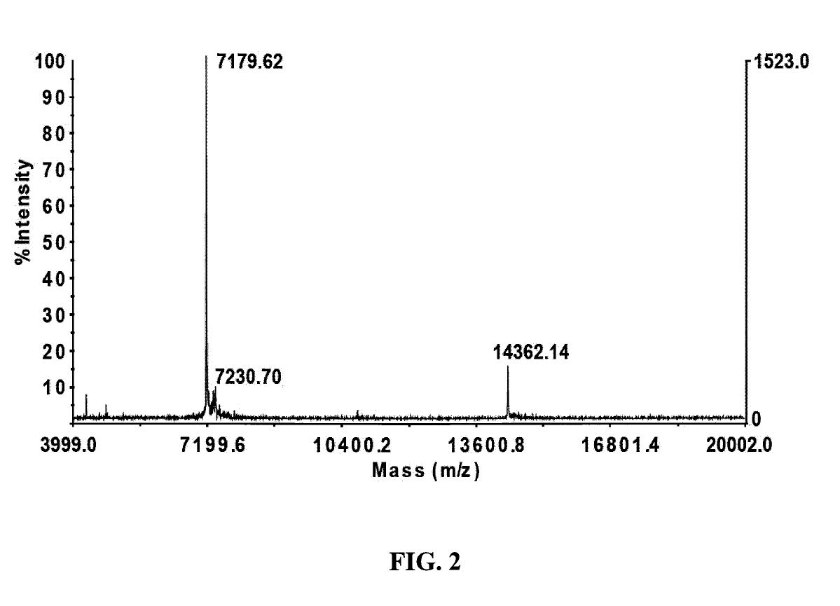

In particular, the present invention also relates to biosurfactants that may be produced by the processes described herein. For example, modifications of conventional fermentation technique by changing the fermentation media and conditions to render the hydrophobin expressed become insoluble in the broth while the fermentation was still in progress prevented foam out during fermentation is presented herein. The composition of the hydrophobin produced using the modified fermentation is presented in FIG. 2 and the peak at mass 7180 corresponds to the full length hydrophobin molecule. Interestingly, the hydrophobin produced by the methods presented herein results in a homogeneous product, unlike naturally occurring hydrophobin which is usually a mixture of two variants. Therefore, the present invention also encompasses any hydrophobin having the spectra depicted in FIG. 2.

Advantageously, the precipitation agent is or includes a salt—ionic compounds that can result from the neutralization reaction of an acid and a base comprised of cation(s) and anion(s), e.g. an ionic compound comprising any suitable anion(s), such as halide(s), e.g., chloride, fluoride bromide, or iodide; a citrate; an acetate; a nitrate (or nitric acid salt), a nitrous acid salt, a carbonate; a sulfate; a phosphate; a sulphamate; a phosphonate; or a sulphamate; and any suitable cation, e.g., ammonium, calcium, a metal or transition metal such as aluminum, iron, magnesium, lithium, potassium or sodium The salt advantageously comprises a polyatomic ion, and more preferably comprises a sulfate salt. The salt may be or comprise ammonium sulfate, calcium sulfate, iron sulfate, magnesium sulfate, potassium sulfate or sodium sulfate. In a particularly advantageous embodiment, the salt is or comprises sodium sulfate. In another particularly advantageous embodiment, the salt is or comprises ammonium sulfate. In other embodiment, the salt may be an acetate salt, a carbonate salt, a chloride salt, a citrate salt, a formate salt, a nitrate salt, or a phosphate salt.

In another embodiment, the precipitation agent is an alcohol. The alcohol may be a monohydric or polyhydric alcohol, such as a monhydric or polyhydric C₁-C₆ alcohol, such as methanol, ethanol or isopropyl alcohol.

In another embodiment, the precipitation agent is a water miscible organic solvent. The solvent may be acetone or a ketone.

In another embodiment, the precipitation agent is a water soluble polymer. The polymer may be polyethylene glycol or a polysaccharide, such as dextran. In another embodiment, the precipitation agent is a cationic polymer, such as but not limited to C581 (Cytec Industries, Woodland Park, N.J. 07424).

In a particularly preferred embodiment, the pH of the culture solution is adjusted dependent on the biosurfactant. For example, if the biosurfactant is hydrophobin, the pH is advantageously about 4.0±0.5. The pH may range from about 3.9±0.5 to about 4.1±0.5, about 3.8±0.5 to about 4.2±0.5, about 3.7±0.5 to about 4.3±0.5, about 3.6±0.5 to about 4.4±0.5, about 3.5±0.5 to about 4.5±0.5, about 3.4±0.5 to about 4.6±0.5, about 3.3±0.5 to about 4.7±0.5, about 3.2±0.5 to about 4.8±0.5, about 3.1±0.5 to about 4.9±0.5, about 3.0±0.5 to about 5.0±0.5, about 2.9±0.5 to about 5.1±0.5, about 2.8±0.5 to about 5.2±0.5, about 2.7±0.5 to about 5.3±0.5, about 2.6±0.5 to about 5.4±0.5, about 2.5±0.5 to about 5.5±0.5, about 2.4±0.5 to about 5.6±0.5, about 2.3±0.5 to about 5.7±0.5, about 2.2±0.5 to about 5.8±0.5, about 2.1±0.5 to about 5.9±0.5 or about 2.0±0.5 to about 6.0±0.5.

If the biosurfactant is rhamnolipid or sophorolipid, the pH is advantageously about 2.5±0.5. The pH may range from about 2.4±0.5 to about 2.6±0.5, about 2.3±0.5 to about 2.7±0.5, about 2.2±0.5 to about 2.8±0.5, about 2.1±0.5 to about 2.9±0.5, about 2.0±0.5 to about 3.0±0.5, about 1.9±0.5 to about 3.1±0.5, about 1.8±0.5 to about 3.2±0.5, about 1.7±0.5 to about 3.3±0.5, about 1.6±0.5 to about 3.4±0.5, about 1.5±0.5 to about 3.5±0.5, about 1.4±0.5 to about 3.6±0.5, about 1.3±0.5 to about 3.7±0.5, about 1.2±0.5 to about 3.8±0.5, about 1.1±0.5 to about 3.9±0.5, about 1.0±0.5 to about 4.0±0.5, about 0.9±0.5 to about 4.1±0.5, about 0.8±0.5 to about 4.2±0.5, about 0.7±0.5 to about 4.3±0.5, about 0.6±0.5 to about 4.4±0.5 or about 0.5±0.5 to about 4.5±0.5.

In another embodiment, the advantageous pH of other surfactants may be about pH 7.0±0.5, about pH 7.1±0.5, about pH 7.2±0.5, about pH 7.3±0.5, about pH 7.4±0.5, about pH 7.5±0.5, about pH 7.6±0.5, about pH 7.7±0.5, about pH 7.8±0.5, about pH 7.9±0.5, about pH 8.0±0.5, about pH 8.1±0.5, about pH 8.2±0.5, about pH 8.3±0.5, about pH 8.4±0.5, about pH 8.5±0.5, about pH 8.6±0.5, about pH 8.7±0.5, about pH 8.8±0.5, about pH 8.9±0.5, about pH 9.0±0.5, about pH 9.1±0.5, about pH 9.2±0.5, about pH 9.3±0.5, about pH 9.4±0.5, about pH 9.5±0.5, about pH 9.6±0.5, about pH 9.7±0.5, about pH 9.8±0.5, about pH 9.9±0.5, about pH 10.0±0.5, about pH 10.1±0.5, about pH 10.2±0.5, about pH 10.3±0.5, about pH 10.4±0.5, about pH 10.5±0.5, about pH 10.6±0.5, about pH 10.7±0.5, about pH 10.8±0.5, about pH 10.9±0.5, about pH 11.0±0.5, about pH 11.1±0.5, about pH 11.2±0.5, about pH 11.3±0.5, about pH 11.4±0.5, about pH 11.5±0.5, about pH 11.6±0.5, about pH 11.7±0.5, about pH 11.8±0.5, about pH 11.9±0.5, about pH 12.0±0.5, about pH 12.1±0.5, about pH 12.2±0.5, about pH 12.3±0.5, about pH 12.4±0.5, about pH 12.5±0.5, about pH 12.6±0.5, about pH 12.7±0.5, about pH 12.8±0.5, about pH 12.9±0.5, about pH 13.0±0.5, about pH 13.1±0.5, about pH 13.2±0.5, about pH 13.3±0.5, about pH 13.4±0.5, about pH 13.5±0.5, about pH 13.6±0.5, about pH 13.7±0.5, about pH 13.8±0.5, or about pH 13.9±0.5.

As mentioned earlier, adjusting of pH need not include carrageenan, and any use of carrageenan need not include pH adjustment, particularly below pH 3.5 or 3. Also, any adjustment of ionic strength to below 0.5, or below 0.4, below or 0.3, or below 0.2 is need not include adjusting pH to below 3.5 or 3 and/or use of carrageenan. pH adjustment that results in decreasing the pH may be achieved be addition of an acid, such as sulfuric acid.

The precipitation agent, e.g., added salt, alcohol, water miscible organic solvent, or water soluble polymer or a cationic polymer, and/or pH adjustment, and/or temperature adjustment and/or temperature increase, is added or pH adjustment performed in amounts to achieve sufficient precipitation or insolubilization of the biosurfactant, e.g., hydrophobin such as hydrophobin II, advantageously to avoid use of antifoam. That is, insolubilization is advantageous for foam control. In other words, insolubilization is performed as the means to control foam, and the amount of precipitation agent or—the amount of pH adjustment or temperature adjustment is such to cause an amount of insolubilization so as to control foaming. Also, it is advantageous that the amount of precipitation agent or amount of pH adjustment or temperature adjustment does not adversely impact upon cell or microorganism growth and/or production of biosurfactant.

The preferred pH range for low solubility of hydrophobin is about 3.5-4.5. For other surfactants, the pH range may be quite different and an optimal pH range may be determined by one of skill in the art.

For hydrophobin, in the pH range between 3.5 and 4.5, the required concentration of ammonium sulfate or of sodium sulfate is temperature dependent. Between about 30° C. and about 60° C., a preferred concentration is about 0.1% to about 5%. At about 30° C. or below, the concentration of sodium sulfate is advantageously above 5%, up to the saturation limit of the salt, which is about 15% for sodium sulfate and about 30-50% for ammonium sulfate, dependent on temperature.

Again, for other biosurfactants, both the temperatures and the concentrations of these precipitants may be quite different and would have to be determined experimentally for each.

In other advantageous embodiments, the biosurfactant may be rhamnolipid, sophorolipd or surfactin. Advantageously, rhamnolipid may be precipitated with sodium chloride, calcium chloride, sodium sulfate and/or a cationic polymer (such as, but not limited to, C581). Advantageously, sophorolipid may be precipitated with sodium chloride, calcium chloride, sodium sulfate and/or a cationic polymer (such as, but not limited to, C581). Advantageously, surfactin may be precipitated with sodium chloride, calcium chloride and/or sodium sulfate. In another advantageous embodiment, rhamnolipid, sophorolipid and surfactin may be propagated in Bacillus licheniformis, Bacillus subtilis and/or Trichoderma reseei.

Salt-free, concentrated solutions of hydrophobin, at or above 80 g per Liter, may be precipitated by very high temperature alone to control foaming. For example, a temperature of 80° C. effectively destroyed any foam that had formed during the heating to that temperature wherein the pH at that temperature was between about 6 and 7.

Hydrophobin may be precipitated with isopropyl alcohol at room temperature. Two to three volumes of isopropanol when added to one volume of hydrophobin solution in water will precipitate hydrophobin.

In another embodiment, the physical condition is temperature.

In a particularly preferred embodiment, the temperature of the culture solution is adjusted. Temperature can ranges here widely depending on biosurfactants and the concentration and may range from about 20° C. to about 90° C. For hydrophobin, the temperature is above 30° C. For rhamnolipid, sophorolipid or surfactin, the temperature may be about 20° C. to about 30° C.

There are several ways to test the effectiveness of foam control. The easiest is examining the surface foam for evidence of significant reduction in total volume. Entrained air can be tested with a similar equipment that have a density meter that can record changes of the liquor density over time.

In an advantageous embodiment, the effectiveness of foam control may be measured by the overrun of a treated solution, which is a calculated value which relates to the volume of a foamed solution minus the starting volume, divided by the starting volume, reported as a fraction or percentage. An overrun of zero means solution contains no foam.

Foam reduction index may also be utilized as a measure of the effectiveness of a treatment for controlling the foam. It is the ratio of the overrun of an untreated solution to a treated solution.

In another embodiment, the effectiveness of foam reduction may also be measuring absolute and relative insolubility of the biosurfactant. Foam reduction may be determined to be effective if the biosurfactant is at least about 5%, 10%, 15%, 20%, 25%, 30%, 35%, 40%, 45%, 50%, 55%, 60%, 65%, 70%, 75%, 80%, 85%, 90% or 95% insoluble.

Foam reduction may be determined to be effective if less than 0.1 g/kg, 0.5 g/kg, 1 g/kg, 2 g/kg, 3 g/kg, 4 g/kg, 5 g/kg, 6 g/kg, 7 g/kg, 8 g/kg, 9 g/kg or 10 g/kg of the biosurfactant (measured in g) is present in solution (measured in kg).

In an advantageous embodiment, foam reduction may be determined to be effective if the biosurfactant is at least about 25% insoluble and/or if no more than 1 g/kg of the biosurfactant is present in the supernatant.

In an advantageous embodiment if the biosurfactant is a protein, the insolubility of the protein may be quantified by measuring the amount of the protein in the precipitate (insoluble) and the supernatant (soluble). The absolute and relative insolubility may be determined by quantifying the protein in the precipitate (insoluble) and the supernatant (soluble). Methods of quantifying proteins are known to one of skill in the art.

Methods of quantifying a non-protein biosurfactants in a precipitate and in solution are well known to one of skill in the art.

Multiple light scattering coupled with vertical scanning is the most widely used technique to monitor the dispersion state of a product, hence identifying and quantifying destabilisation phenomena [Roland et al. International Journal of Pharmaceutics 263 (2003) 85-94, Lemarchandet al. Pharmaceutical Research, 20-8 (2003) 1284-1292, Mengual et al. Colloids and Surfaces A: Physicochemical and Engineering Aspects 152 (1999) 111-123, Bru et al. Particle sizing and characterisation Ed T. Provder and J. Texter (2004)). It works on any concentrated dispersions without dilution, including foams. When light is sent through the sample, it is backscattered by the bubbles. The backscattering intensity is directly proportional to the size and volume fraction of the dispersed phase. Therefore, local changes in concentration (drainage, syneresis) and global changes in size (ripening, coalescence) are detected and monitored. Conductivity can also be used to monitor concentrations of ingredients in a growth media, as well as turbidity.

A particular advantage from the present invention is that the process for producing a biosurfactant can be continuous. For instance, in the practice of the invention the bioreactor or fermenter can have means for removing solubilized biosurfactant, e.g., hydrophobin, for instance, a valve-controlled fluid conduit from which solubilized biosurfactant can be removed from the bioreactor or fermenter. The valve can be operated in connection with processor or microprocessor for the opening and closing of the valve. The processor or microprocessor can receive a signal from a sensor, such as a sensor that indicates concentration or change thereof of biosurfactant in solution or turbidity of solution or another parameter, such as amount of foam and based on that sensor signal the processor or microprocessor can indicate the opening or closing of the valve for removing solubilized biosurfactant; or the microprocessor or processor can cause the opening or closing of the valve based on other parameters, such as time from when precipitation agent and/or precipitation condition was added or applied, achievement of concentration of precipitation agent and/or achievement of precipitation condition, including over a period of time. The bioreactor or fermenter can also include means for adding a precipitation agent or fluid or other condition to achieve precipitation condition, e.g., valve-controlled fluid conduit by which can be added a precipitation agent, for instance, a salt, advantageously in a solution, an alcohol, or a fluid that achieves precipitation condition, e.g., acid to reduce pH, or a heater. The valve or heater can be in connection with processor or microprocessor for the opening and closing of the valve or turning on or off the heater. The processor or microprocessor can receives a signal from a sensor, such as a sensor that indicates concentration or change thereof of biosurfactant in solution or another parameter such as foam and based on that sensor signal the processor or microprocessor can indicate the opening or closing of the valve or turning on or off of the heater for adding precipitation agent or fluid or other means for causing solubilization; or the microprocessor or processor can cause the opening or closing of the valve based on other parameters, such as time from when solubilized biosurfactant removed. Further the bioreactor or fermenter can include means for adding media and/or cells or microorganisms or other ingredients of media producing biosurfactant. Inevitably in removal of solubilized biosurfactant, some media, and/or cells or microorganisms or other ingredients of the media producing biosurfactant will be lost with the solubilized surfactant, and the bioreactor or fermenter includes means to replenish. This replenishing means can for instance be valve-controlled fluid connection means from which cells or organisms or media or other ingredients of media are fed to the bioreactor or fermenter. The valve can be in connection with processor or microprocessor for the opening and closing of the valve. The processor or microprocessor can receives a signal from a sensor, such as a sensor that indicates concentration or change thereof of cells or microorganisms or other ingredients of media or turbidity of solution or another parameter, and based on that sensor signal the processor or microprocessor can indicate the opening or closing of the valve for replenishing; or the microprocessor or processor can cause the opening or closing of the valve based on other parameters, such as time. When cells, microorganisms or media or ingredients of media are harvested with solubilized surfactant, such cells, microorganisms or media or ingredients of media can be separated from the solubilized biosurfactant and recycled back to the fermenter or bioreactor, e.g., via the replenishing means. The sensors of the foregoing discussion can be one or more sensor in or in connection with the bioreactor or fermenter.

In this fashion, media for producing and that produces the biosurfactant, e.g., hydrophobin such as hydrophobin H, rhamnolipid, sophorolipid or surfactin, is fed to the bioreactor or fermenter, as foam occurs or is occurring or before it significantly occurs or after a time that the media is in the bioreactor or fermenter, a precipitation agent or precipitation condition is added or applied, e.g., sodium sulfate is added and/or alcohol is added and/or heat applied and/or pH adjusted, advantageously downward, whereby foam is controlled and the biosurfactant precipitates or insolubilizes. Insolubilized biosurfactant is removed from the bioreactor or fermenter. And media or ingredients thereof, e.g., cells or microorganisms, nutrients, or other ingredients of the media, are fed into the bioreactor or fermenter, i.e. there is a replenishing of media or ingredients thereof, e.g., cells or microorganisms, nutrients, or other ingredients of the media. Optionally, media or ingredients thereof, e.g., cells or microorganisms, nutrients, or other ingredients of the media, that come off with the insolubilized biosurfactant are recycled back to the bioreactor or fermenter. There thus can be continuous production of a biosurfactant.

The method may be conducted in a reactor, for example a bioreactor. As used herein, “bioreactor” refers to any manufactured or engineered device or system capable of supporting a biologically active environment. For example, a bioreactor may include a vessel in which one or more chemical and/or biological processes occurs. In some embodiments, these processes involve organisms or biochemically active substances derived from such organisms. In some embodiments, organisms or cells may be grown in the bioreactor. In some embodiments, organisms may be suspended or immobilized in the reactor during use.

Reactors utilized in conjunction with this method may include, but are not limited to batch reactors, fed batch reactors, continuous reactors, such as continuously stirred tank reactors, moving media, packed bed, fibrous bed, membrane reactors or any other systems known or yet to be discovered in the art.

In some embodiments, use of a continuous reactor, allows materials to be continuously pumped through the reactor. The flow of materials pumped may promote mixing. In some embodiments, static mixers, such as baffles, and/or mechanical agitation may be used in a reactor to promote mixing of the components.

In some embodiments, the method may be conducted using a bioreactor. Cells and media may be provided to bioreactor via inputs including, but not limited to ports, pipes, tubes, hoses, and/or any other input device known in the art. Multiple inputs may be used to provide the cells, media, and/or nutrients to the reactor.

A control system including one or more sensors, and one or more controllers may be utilized to control conditions within the reactor. Controllers may include, but are not limited to processors, microprocessors or other controllers known in the art. Information utilized to control the reactor conditions may be provided to the controllers from one or more sensors and/or from a user.

Sensors may be utilized to measure conditions within the reactor, including but not limited to temperature, pH, composition, presence of foam, an amount of foam, pressure, presence of precipitate, an amount of precipitate and/or any other relevant measurement known in the art. Multiple sensors may positioned around the reactor to determine conditions at specific locations. For example, a sensor to determine an amount of or the presence of precipitate may be positioned proximate the bottom of the reactor in some embodiments. Embodiments may include sensors to determine the presence of foam proximate input openings, various positions within the tank and/or any position of interest. Any sensor known in the art may be used.

Some embodiments may include windows or openings in tank for observation. Some reactors may include lights positioned in the reactor to for observation of conditions within the reactor. An operator may be able to observe conditions in tank and input data into a user interface connected to one or more controllers to adjust conditions within the tank.

For example, based on data from sensors and/or user input valves may be opened or closed based on needs in the reactor. In some embodiments, valves on inputs may control addition of nutrients, buffer, media, organisms and/or other components.

Some embodiments may include allowing the cells to grow within the inner chamber of the reactor. Nutrients, media and cells may be added to the reactor in a ratio sufficient to optimize growth of an organism of interest. In some embodiments, the composition of the added materials is controlled to optimize production of a component of interest. For example, a component of interest may be a protein or a compound.

In some embodiment, as the component of interest increases in concentration foaming may begin to occur. Windows and/or sensors may be utilized to detect foaming in the reactor. For example, a sensor or window may be used to determine if foaming is occurring. Once foaming is detected, the controller may direct that a precipitating agent be added to the reactor. In some embodiments, the precipitating agent may allow the component of interest to precipitate out of the solution. The precipitated component may accumulate at the bottom of reactor.

Some embodiments may include one or more sensors positioned proximate the bottom of the reactor to determine whether precipitate is present and/or the quantity of precipitate present. These sensors may communicate with one or more controllers. A controller may use this input to determine to open a valve proximate the bottom of the reactor so that precipitate exits the reactor.

In some embodiments, pumps be utilized along the inputs and outputs to facilitate the movement of materials in the inputs and outputs.

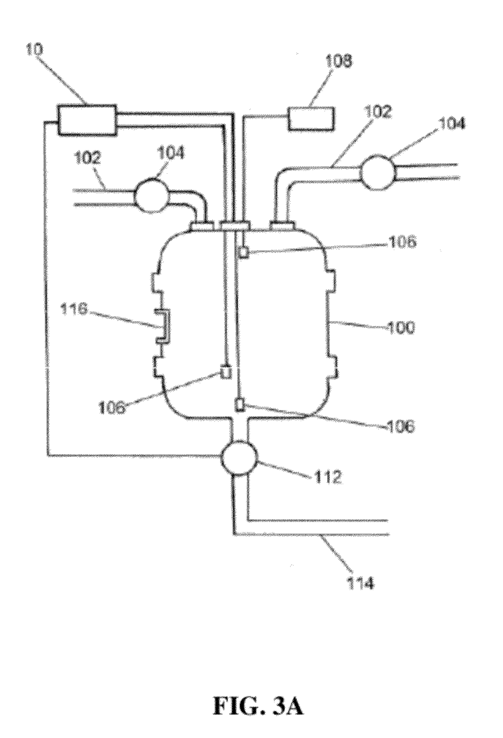

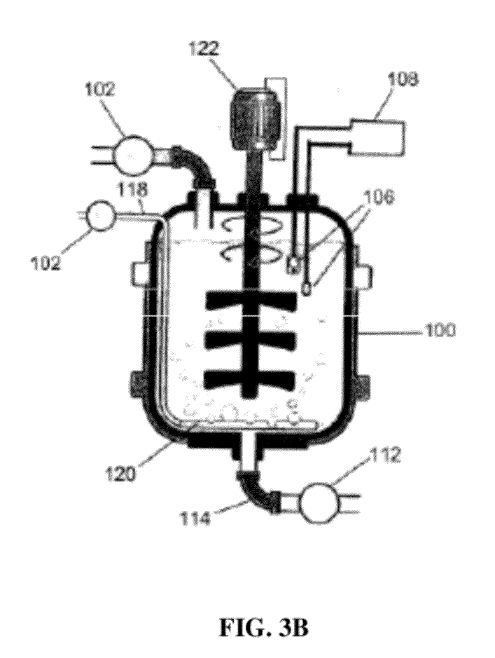

As shown in FIG. 3, some embodiments may include performing the method utilizing reactor 100. Cells, media, and/or nutrients may be provided to reactor 100 via inputs 102. As shown in FIG. 3, input 102 may include valve 104 used to control the delivery of organisms and/or media to the vessel. In some embodiments, multiple inputs may be utilized to deliver organisms and/or media to different locations of the reactor. In some embodiments, as depicted in FIG. 3, cells and media are provided via input 102. Multiple sensors 106 may be positioned at locations throughout reactor 100. Sensor 106 provide data to controllers 108, 110. Controllers 108, 110 are capable of controlling an amount of cells, media, nutrients, precipitating agent and/or other components. In some embodiments, controllers may make adjustments to control conditions in the reactor, the inputs, and/or the outputs.

Some embodiments may include allowing the cells to grow within the inner chamber of the reactor. As the component of interest increases in concentration foaming may begin to occur. In some embodiments, windows and/or sensors may be utilized to detect foaming in the reactor. Once foaming is detected, a precipitating agent may be added to the reactor. In some embodiments, the precipitating agent may allow the component of interest to precipitate out of the solution. The precipitated component may detected using sensors 106. In some embodiments a window 116 may be present in reactor 100 to allow a user to observe conditions in the reactor.

Controller 108 is connected to output valve 112. Controller 110 may direct valve 112 to open to allow precipitate to leave the tank via output 114. In some embodiments, user input may allow control to direct valve 112 to open and/or close as needed.

As shown in FIG. 3, nutrients including, but not limited to air, oxygen or any other nutrients known in the art may be provided to reactor using input 118. Input 118 may be coupled to delivery device 120 to provide nutrients to reactor 100. In some embodiments, the delivery device may be positioned at any location in the reactor. Some embodiments include mixer 122 to promote mixing of the components in the reactor.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Clarified Unpurified Hydrophobin Solution

A method for reducing foam formation in a clarified hydrophobin solution using sodium sulfate and pH adjustment is presented herein. The hydrophobin solution was obtained using conventional production methods. The concentration of the hydrophobin solution was 33 g/kg. The sodium sulfate treatment was achieved by adding anhydrous sodium sulfate to reach a final concentration of 2.5% w/w with gentle mixing and allowed to dissolve. The pH was adjusted to 4.0 using 1% sulfuric acid. The solution was mixed at 10° C. for 16 hr. 2×5 mL of the Na2SO4 treated concentrate was centrifuged to remove the liquid portion. Each of the precipitates was resuspended to the same volume as the initial Hydrophobin concentrate in water. A spatula was used to loosen and resuspend the precipitates. 2×5 mL of untreated Hydrophobin concentrate was prepared. One of the concentrates and one of the Na₂SO₄ treated concentrates were mixed by shaking.

A picture was taken and the total volume of each tube was recorded immediately and after 4 hr. The results are presented in Table 1. The sodium sulfate treated solution has a soluble hydrophobin concentration of 1 g/L. 97% of the hydrophobin is insoluble after the sodium sulfate addition.

TABLE 1

| | | Post Mixing Volume. | | Overrun. | |
|---|---|---|---|---|---|
| | Initial | After holding For | | After holding for | |
| | Volume | (mL) | | (%) | |
| Treatment | (mL) | 0 hr | 4 hr | 0 hr | 4 hr |
| None | 5 | 14 | 14 | 180% | 180% |
| Sodium Sulfate | 5 | 6.5 | 6.5 | 30% | 30% |
| Treated | | | | | |

| | | |
|---|---|---|
| Foam Reduction Index | 6.0 | 6.0 |

### Example 2

### Purified Hydrophobin Solution

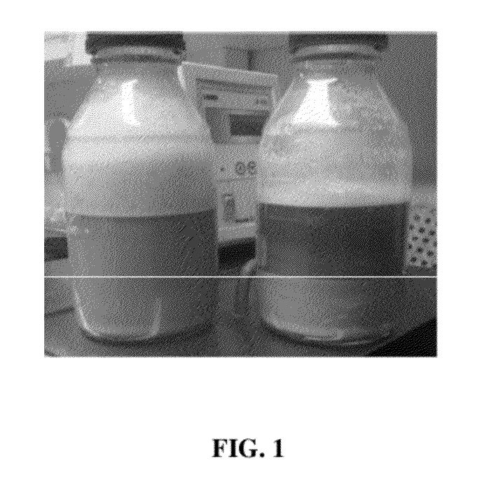

A method for reducing foam formation of hydrophobin solution using heat is herein presented. The hydrophobin solution has a concentration of 130 g/kg. When 320 g of hydrophobin solution in a 500 mL Pyrex was mixed, foam filled the headspace of the bottle (picture on left, FIG. 1). When another similarly mixed hydrophobin solution was heated to 80° C., sediments formed and the foam collapsed (picture on right, FIG. 1). The results are presented in Table 2.

TABLE 2

| Treatment | Initial Volume (mL) | After Treatment (mL) | Overrun (%) |
|---|---|---|---|
| None | 320 | >500 | >56% |
| Heat Treated | 320 | 350 | 9% |

| | |
|---|---|
| Foam Reduction Index | >6.2 |

### Example 3

### Fermentation Using Conventional Technique

Table 3 describes the broth appearances of broth when a conventional approach for fermenting Trichoderma reseei expressing either recombinant cellulase or a recombinant hydrophobin. The fermentation media and conditions and the harvest procedure were the same. At the end of the fermentation, the target molecules being expressed were fully soluble in both cases. Table 3 shows the results.

TABLE 3

| | Cellulase | Hydrophobin |
|---|---|---|
| Antifoam Consumption | 0.3 g/kg- | 11.4 g/kg- |
| | harvest broth | harvest broth |
| Foam out during fermentation | None | Yes |
| Overrun | 0% | 240% |

### Example 4

### Hydrophobin Fermentation Broth Foam Reduction

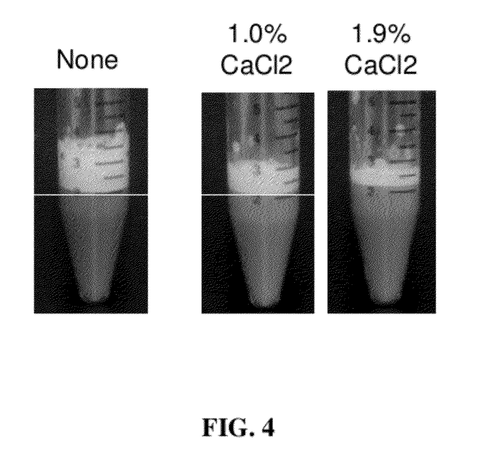

The use of sodium sulfate for reducing foam in a fermentation broth prepared by culturing Trichoderma reseei that expressed recombinant hydrophobin using conventional fermentation and harvest techniques, as depicted in FIGS. 3 and 4, is presented herein.

The harvest broth was treated with 2.5% sodium sulfate and the pH was adjusted to 3.9 with 10% sulfuric acid at 28° C. over 2 hours, and stored at 10° C. The treated broth has 0.2 g/kg of soluble hydrophobin.

### Example 5

### Hydrophobin Fermentation Broth Foam Reduction

The use of ammonium sulfate for reducing foam in a fermentation broth prepared by culturing Trichoderma reseei that expressed recombinant hydrophobin using conventional fermentation and harvest techniques is described below.

The harvest broth was treated with 5% ammonium sulfate at 22° C. The resulting broth did not contain any foam after treatment, contains needle shaped hydrophobin crystals.

### Example 5

### Foam Control During Hydrophobin Fermentation Harvest

A method for controlling foam during the harvest of a conventionally fermented Trichoderma reseei broth that expressed recombinant hydrophobin is presented herein. Foam out problems associated with conventional method for fermenting hydrophobin are exacerbated during harvest. During harvest, the pressurized contents of the fermentor must be brought back to ambient pressure, which leads to outgassing of the dissolved air. Surprisingly, this propensity to foam can be effectively controlled by adding precipitating agents to fermentation broth, specifically, sodium sulfate. The precipitation of hydrophobin in the broth reduces the foaming to a point where it is controllable even during depressurization.

At the end of the fermentation (referred to as “End of Fermentation Broth”), the fermentor operating parameters were changed as follows: airflow to redirect from bottom feed into the sparger to feeding into the headspace of the fermentor, pressure to remain at 20 psig, temperature to remain at 28° C., and agitation to remain at 160 rpm. Sodium sulfate stock solution at 15% w/w Na₂SO₄ and at pH 2.8 was pumped into the fermentor at a rate at of 6 liters per minute until the resulting broth had reached a Na₂SO₄=concentration of 2.5%. The resulting broth had a pH of 4 (referred to as “Na₂SO₄/pH 4 Before Depressurized Broth”). Then the fermentor was slowly depressurized by reducing the airflow from 1600 LPM to 100 LPM and at the same time lowering the pressure from 20 psig to 0 psig, both linearly over 1 hour. The broth is referred to as “Na₂SO₄/pH 4 Depressurized Broth”. After de-pressurization, the broth was kept in the fermentor at 28° C., with mixing on while the pH was monitored and adjusted to pH 4 until no change in pH was observed. The broth is referred to as “Na₂SO₄/pH 4 Harvest Broth”.

Table 4 shows the results of the physical appearance of broth sample taken during the various stages of the harvest treatment. The treatment increased the density of the broth from 0.605 g/mL to 1.042 g/m. The overrun is calculated using starting weight. The treated broth soluble hydrophobin concentration is 0.2 g/kg, about 26-fold lower than that of the untreated broth.

TABLE 4

| | | | #2 | | |
|---|---|---|---|---|---|
| | | #1 | Na₂SO_{4/}pH 4 | #3 | #4 |
| | | End of | Before | Na₂SO₄/pH 4 | Na₂SO₄/pH 4 |
| | Unit | Fermentation | Depressurized | Depressurized | Harvest |
| Broth Weight | g | 99.80 | 99.80 | 100.00 | 100.00 |
| Broth Volume | mL | 165 | 120 | 102 | 102 |
| Density | g/mL | 0.605 | 0.832 | 0.980 | 0.980 |
| Overun | % | 65% | 20% | 2% | 2% |
| Foam Reduction Index | | | 3.2 | 32.7 | 32.7 |

### Example 7

### Foam Control During Hydrophobin Fermentation

The modifications of conventional fermentation technique by changing the fermentation media and conditions to render the hydrophobin expressed become insoluble in the broth while the fermentation was still in progress prevented foam out during fermentation is presented herein. Table 5 shows the modifications and the results. The concentrations of hydrophobin in the supernatant of the harvest broths for all the runs with modification were less than 0.5 g/kg.

TABLE 5

| | Conventional | | | Modified | | |
|---|---|---|---|---|---|---|
| Ammonium sulfate (g/kg) | 4.3 | 4.3 | 4.3 | 25.0 | 25.0 | 25.0 |
| Fermentation pH | 4.5 | 4.5 | 4.5 | 4.0 | 4.0 | 4.0 |
| Foam Out During | Yes | Yes | Yes | None | None | None |
| Fermentation | | | | | | |
| Harvest Broth Antifoam | 10.4 | >5.9 | >11.4 | 4.4 | 5.5 | 5.4 |
| (g/kg) | | | | | | |

### Example 8

### Antifoam Usage Reduction During Hydrophobin Fermentation

The modifications of conventional fermentation technique by changing the fermentation media and conditions to render the hydrophobin expressed become insoluble in the broth while the fermentation was still in progress reduced the amount of antifoam required to prevent foam out are presented herein.

33 g/kg of antifoam was measured in a conventional fermentation run, 6.1-7.5-fold higher than the modified fermentation shown above in “Foam Control during Hydrophobin Fermentation”.

### Example 9

### Hydrophobin Composition

The composition of the hydrophobin produced using the modified fermentation is presented in FIG. 2. Peak at mass 7180 corresponds to the full length hydrophobin molecule.

### Example 10

### Foam Reduction in Rhamnolipid Clarified Solution

The reduction in foam formation in a clarified rhamnolipid (Product JBR515 Lot #. 110321, gift from Jeneil Biosurfactant Co., LLC, 400 N. Dekora Woods Blvd, Saukville, Wis. 53080) solution was measured following pH adjustment, sodium chloride, calcium chloride, sodium sulfate and cationic polymer C581 (Cytec Industries, Woodland Park, N.J. 07424) treatments. The rhamnolipid solution was prepared by adding 0.21 grams of JBR515 to 93 grams of de-ionized water, and mixed gently for 5 minutes.

To test reduction in foam formation, 5 grams of the prepared solution was transferred to a clear 15-mL conical tube, the treatment chemical added, and the tube mixed gently by inversion until the chemical was dispersed or dissolved. The treated solution and an untreated solution were shaken 20 times and the appearances of the samples were captured immediately using digital camera. The appearances of the liquid portion of the samples were assessed by visual inspection against the untreated sample. The volume occupied by each shaken solution was recorded and Overrun and Foam Reduction Index were calculated and are shown in Table 6. Turbidity measurements were made using a HACH 2100AN Turbidimeter (Hach Company, Loveland, Colo.) and are reported as NTU (Nephelometric Turbidity Units) values in Table 6.

TABLE 6
Treatment conditions and corresponding Overrun, Foam Reduction Index and
turbidity in Rhamnolipid Clarified Solution

| | Treatments | | | | | | | | | |
|---|---|---|---|---|---|---|---|---|---|---|
| | | pH 2.75 | | | | | | | | |
| | | (with | | | | 2.0% | | 4.8% | | |
| | | sulfuric | | 1.0% | | Calcium | | Sodium | | 1.0% |
| Results | None | acid) | None | NaCl | None | Chloride | None | Sulphate | None | C581 |
| Overrun | 160% | 36% | 150% | 104% | 180% | 42% | 180% | 30% | 160% | 120% |
| Turbidity | 0.468 | 10.8 | 0.468 | 1.89 | 0.468 | 16.9 | 0.468 | 3.02 | 0.468 | 11.3 |
| (NTU) | | | | | | | | | | |

| | | | | | |
|---|---|---|---|---|---|
| Foam | 4.4 | 1.4 | 4.3 | 6.0 | 1.3 |
| Reduction | | | | | |
| Index | | | | | |

### Example 11

### Foam Reduction in Sophorolipid Clarified Solution

The reduction in foam formation in a clarified sophorolipid (Product SO_SOPHS Lot#10175A, SoliancE, Route de Bazancourt 51110 Pomacle, France) solution was measured following pH adjustment, sodium chloride, calcium chloride, sodium sulfate and cationic polymer C581 treatments. The sophorolipid solution was prepared by adding 0.28 grams of SO_SOPHS to 122 grams of de-ionized water, and pH adjusted to 10.1 using 1 N.NaOH. The solution was mixed gently during pH adjustment. Reduction in foam formation was measured as described in the Rhamnolipid Clarified Solution section. The volume occupied by each shaken solution was recorded and Overrun and Foam Reduction Index were calculated and are shown in Table 7. Turbidity measurements were made using a HACH 2100AN Turbidimeter (Hach Company, Loveland, Colo.) and are reported as NTU (Nephelometric Turbidity Units) values in Table 7.

TABLE 7
Treatment conditions and corresponding Overrun and Foam Reduction Index and turbidity in
Sophorolipid Clarified Solution

| | Treatments | | | | | | | | | |
|---|---|---|---|---|---|---|---|---|---|---|
| | | pH 2.5 | | | | | | | | |
| | | (with | | 3.9% | | 0.9% | | 4.2% | | |
| | | sulfuric | | sodium | | Calcium | | Sodium | | 1.0% |
| Results | None | acid) | None | chloride | None | Chloride | None | Sulphate | None | C581 |
| Overrun | 160% | 2% | 130% | 40% | 160% | 10% | 150% | 90% | 140% | 22% |
| Turbidity | 0.434 | Cloudy | 0.43 | 1.13 | 0.43 | 15.00 | 0.43 | 1.23 | 0.43 | 3.83 |
| (NTU) | | with | | | | | | | | |
| | | precipitates | | | | | | | | |

| | | | | | |
|---|---|---|---|---|---|
| Foam | 80.0 | 3.3 | 16.0 | 1.7 | 6.4 |
| Reduction | | | | | |
| Index | | | | | |

### Example 12

### Foam Reduction in Surfactin Clarified Solution

The reduction in foam formation in a clarified surfactin (Part # S3523-50MG, Sigma Alrich, P.O. Box 951524 Dallas, Tex. 75395-1524) solution was measured following pH adjustment, sodium chloride; calcium chloride, and sodium sulfate treatments. Surfactin stock solution was prepared by adding 2.03 grams of de-ionized water directly to the vial containing surfactin and pH was adjusted between 6-7 (as measured by pH strip paper) using 1N NaOH. The stock solution was further diluted by adding 8.9 g of de-ionized water to 0.79 g of the stock solution. Reduction in foam formation was measured as described in Rhamnolipid Clarified Solution section. The appearances of the liquid portion of the samples were assessed by visual inspection against the untreated sample. Table 8 shows the Overrun, Foam Reduction Index, and appearance of the liquid portion for each of the treatments performed.

TABLE 8
Treatment conditions and corresponding Overrun, Foam Reduction Index and
appearance of liquid portion in Surfactin Clarified Solution

| | Treatments | | | | | | | |
|---|---|---|---|---|---|---|---|---|
| | | pH 2.5 | | | | | | |
| | | (with | | 3.0% | | 0.9% | | 2.9% |
| | | sulfuric | | sodium | | Calcium | | Sodium |
| Results | None | acid) | None | chloride | None | Chloride | None | Sulphate |
| Overrun | 50% | 25% | 55% | 10% | 63% | 13% | 50% | 24% |
| Liquid | Clear | Cloudy | Clear | Cloudy | Clear | Cloudy | Clear | Cloudy |
| portion | | with | | | | with | | |
| appearance | | particulates | | | | particulates | | |

| | | | | |
|---|---|---|---|---|
| Foam | 2.0 | 5.5 | 5.0 | 2.1 |
| Reduction | | | | |
| Index | | | | |

Table 9 shows the Overrun, Foam reduction Index and appearance of liquid portion for treated and untreated solutions that were kept at room temperature for 0.5 hr.

TABLE 9
Treatment conditions and corresponding Overrun, Foam Reduction
Index and appearance of liquid portion in Surfactin Clarified
Solution after incubation at room temperature for 0.5 hr.

| | Treatments | | | | |
|---|---|---|---|---|---|
| | | pH 2.5 (with | 3.0% sodium | 0.9% Calcium | 2.9% Sodium |
| Results | None | sulfuric acid) | chloride | Chloride | Sulphate |
| Overrun | 40% | 5% | 2% | 2% | 6% |
| Liquid portion | Clear | Cloudy with | Cloudy with | Very Cloudy | Cloudy |
| appearance | | particulates | particulates | with particulates | |
| Foam Reduction | — | 8.0 | 16.0 | 16.0 | 7.2 |
| Index | | | | | |

### Example 13

### Foam Reduction in Bacillus licheniformis Fermentation Broth Containing Rhamnolipid

The reduction in foam formation in a Bacillus licheniformis fermentation broth containing rhamnolipid (described in Example 10) was measured following pH adjustment, sodium chloride, calcium chloride, sodium sulfate and cationic polymer C581 treatments. 5.65 grams of JBR515 were added to 100 grams of Bacillus licheniformis fermentation broth produced using techniques known in the art, and the solution mixed gently for 5 minutes. The solution of the resulting broth has a pH of 6.52. Reduction in foam formation was measured as described in the Rhamnolipid Clarified Solution section. Table 10 shows the Overrun and Foam Reduction Index for each of the treatments performed.

TABLE 10
Treatment conditions and corresponding Overrun and Foam
Reduction Index in Rhamnolipid-containing Bacillus licheniformis
fermentation broth.

| | Treatments | | | | | |
|---|---|---|---|---|---|---|
| | | pH 4.62 | | | | |
| | | (with | 1.0% | 2% | 5% | |
| | | sulfuric | sodium | Calcium | Sodium | |
| Results | None | acid) | chloride | Chloride | Sulphate | 3% C581 |
| Overrun | 90% | 25% | 80% | 14% | 58% | 23% |
| Foam | — | 3.6 | 1.1 | 6.4 | 1.6 | 4.0 |
| Reduction | | | | | | |
| Index | | | | | | |

### Example 14

### Rhamnolipid in Trichoderma reseei Fermentation Broth Containing Rhamnolipid

The reduction in foam formation in a Trichoderma reseei fermentation broth containing rhamnolipid (described in Example 10) was measured following pH adjustment from the starting solution and/or sodium chloride, sodium sulfate and cationic polymer C581 treatments. 6.53 grams of JBR515 were added to 28 grams of de-ionized water and 100 grams of Trichoderma reseei fermentation broth produced using techniques known in the art, pH adjusted to 6.15 and mixed gently for 5 minutes. Reduction in foam formation was measured as described in Rhamnolipid Clarified Solution section. The reduction in foam formation was measured immediately as well as 30 minutes, therefore there is also retention of reduced foaming. Table 11 shows the Overrun and Foam Reduction Index for each of the treatments performed.

TABLE 11
Treatment conditions and corresponding Overrun
and Foam Reduction Index in Rhamnolipid-containing
Trichoderma reseei fermentation broth

| | | Immediately after shaken | | 0.5 h after shaken | |
|---|---|---|---|---|---|
| | | | Foam | | Foam |
| | | | Reduction | | Reduction |
| Treatment | pH | Overrun | Index | Overrun | Index |
| None | 6.15 | 42% | — | 33% | — |
| sulfuric acid | 4.88 | 8% | 5.3 | 42% | 13.1 |
| 2.7% Sodium | 6.28 | 10% | 4.2 | 3% | 4.2 |
| chloride | | | | | |
| 2.1% calcium | 5.39 | 20% | 2.1 | 10% | 2.1 |
| chloride | | | | | |
| 2.6% sodium | 5.72 | 13% | 3.2 | 20% | 3.2 |
| sulfate + | | | | | |
| sulfuric acid | | | | | |
| 2.2% C581 | 4.26 | 10% | 4.0 | 13% | 4.0 |
| and sulfuric | | | | | |
| acid | | | | | |

### Example 15

### Foam Reduction in Bacillus subtilis Fermentation Broth Containing Rhamnolipid

The reduction in foam formation in a Bacillus subtilis fermentation broth containing rhamnolipid (described in Rhamnolipid Clarified Solution section) was measured following pH adjustment and/or sodium chloride, calcium chloride, sodium sulfate and cationic polymer C581 treatments. 2.71 grams of JBR515 were added to 40.1 grams of Bacillus subtilis fermentation broth produced using techniques known in the art, and the solution mixed gently for 5 minutes. Reduction in foam formation was measured as described in Rhamnolipid Clarified Solution section. Table 12 shows the Overrun and Foam Reduction Index for each of the treatments performed.

TABLE 12
Treatment conditions and corresponding Overrun
and Foam Reduction Index in Rhamnolipid-containing
Bacillus subtilis fermentation broth

| | | Immediately after shaken | | 0.5 h after shaken | |
|---|---|---|---|---|---|
| | | | Foam | | Foam |
| | | | Reduction | | Reduction |
| Treatment | pH | Overrun | Index | Overrun | Index |
| None | 7.4 | 30% | — | 40% | — |
| sulfuric acid | 3.3 | 2% | 22.4 | 2% | 22.4 |
| 1.2% Sodium | 4.66 | 2% | 23.2 | 2% | 23.2 |
| chloride and | | | | | |
| sulfuric acid | | | | | |
| 1.8% calcium | 4.03 | 42% | 0.9 | 6% | 6.9 |
| chloride and | | | | | |
| sulfuric acid | | | | | |
| 2.7% sodium | 4.4 | 7% | 5.6 | 5% | 7.5 |
| sulfate + | | | | | |
| sulfuric acid | | | | | |
| 2.4% C581 | 7.4 | 13% | 3.0 | 2% | 26.3 |

### Example 16

### Foam Reduction in Bacillus licheniformis Fermentation Broth Containing Sophorolipid

The reduction in foam formation in a Bacillus licheniformis fermentation broth containing sophorolipid (described in Sophorolipid Clarified Solution section) solution was measured using pH adjustment, sodium chloride, calcium chloride, sodium sulfate and cationic polymer C581 treatments. 7.63 grams of SO_SOPHS were added to 102.2 grams of Bacillus licheniformis fermentation broth produced using techniques known in the art, and the solution mixed gently for 5 minutes. The pH of the resulting broth was adjusted to 7.23. Reduction in foam formation was measured as described in Rhamnolipid Clarified Solution section. Table 13 shows the Overrun and Foam Reduction Index for each of the treatments performed.

TABLE 13
Treatment conditions and corresponding Overrun and Foam
Reduction Index in Bacillus licheniformis fermentation
broth containing sophorolipid

| | Treatments | | | | | |
|---|---|---|---|---|---|---|
| | | pH 5.2 | | | | |
| | | (with | 4.0% | 1.0% | 5.1% | |
| | | sulfuric | sodium | Calcium | Sodium | 2.7% |
| Results | None | acid) | chloride | Chloride | Sulphate | C581 |
| Overrun | 36% | 6% | 8% | 10% | 24% | 32% |
| Foam | — | 6.2 | 4.5 | 3.6 | 1.5 | 1.1 |
| Reduction | | | | | | |
| Index | | | | | | |

### Example 17

### Foam Reduction in Trichoderma reseei Fermentation Broth Containing Sophorolipid

The reduction in foam formation in a T. reseei fermentation broth containing sophorolipid (described in Sophorolipid Clarified Solution section) solution was measured using pH adjustment and/or, sodium chloride, calcium chloride, sodium sulfate and cationic polymer C581 treatments. 5.5 grams of SO_SOPHS were added to 28 grams of de-ionized water and 100.2 grams of T. reseei fermentation broth produced using techniques known in the art, and the solutions mixed gently for 5 minutes. Reduction in foam formation was measured as described in Rhamnolipid Clarified Solution section. Table 14 shows the Overrun and Foam Reduction Index for each of the treatments performed (ND-not determined).

TABLE 14
Treatment conditions and corresponding Overrun and Foam Reduction
Index in sophorolipid - containing T. reseei fermentation broth

| | | Immediately after shaken | | 0.5 h after shaken | |
|---|---|---|---|---|---|
| | | | Foam | | Foam |
| | | | Reduction | | Reduction |
| Treatment | pH | Overrun | Index | Overrun | Index |
| None | 7.12 | 42% | — | 25% | — |
| sulfuric acid | 4.24 | ND | ND | 42% | ND |
| 4.7% Sodium | 6.7 | 20% | 2.1 | ND | 2.1 |
| chloride | | | | | |
| 3.4% calcium | 5.85 | 2% | 25.0 | 20% | 25.0 |
| chloride | | | | | |
| 3.4% sodium | 4.37 | 31% | 1.4 | 2% | 1.8 |
| sulfate + | | | | | |
| sulfuric acid | | | | | |
| 2.2% C581 | 6.8 | 30% | 1.4 | 23% | 1.9 |

### Example 18

### Foam Reduction in Bacillus subtilis Fermentation Broth Containing Sophorolipid

The reduction in foam formation in a Bacillus subtilis fermentation broth containing sophorolipid (described in Sophorolipid Clarified Solution section) solution was measured using pH adjustment and/or sodium chloride, calcium chloride, sodium sulfate and cationic polymer C581 treatments. 2.61 grams of SO_SOPHS was added to 40.6 grams of B. subtilis fermentation broth produced using techniques known in the art, and the solution mixed gently for 5 minutes. The pH of the resulting broth was 7.27. Reduction in foam formation was measured as described in Rhamnolipid Clarified Solution section. Table 15 shows the Overrun and Foam Reduction Index for each of the treatments performed.

TABLE 15
Treatment conditions and corresponding Overrun
and Foam Reduction Index in sophorolipid containing
Bacillus subtilis fermentation broth

| | | Immediately after shaken | | 2.3 h after shaken | |
|---|---|---|---|---|---|
| | | | Foam | | Foam |
| | | | Reduction | | Reduction |
| Treatment | pH | Overrun | Index | Overrun | Index |
| None | 7.3 | 22% | — | 20% | — |
| sulfuric acid | 2.71 | 17% | 1.2 | 2% | 11.6 |
| 3.0% Sodium | 5.4 | 19% | 1.0 | 2% | 10.4 |
| chloride and | | | | | |
| sulfuric acid | | | | | |
| 1.2% calcium | 6.05 | 10% | 2.0 | 2% | 10.0 |
| chloride | | | | | |
| 3.2% sodium | 5.67 | 25% | 0.8 | 6% | 3.5 |
| sulfate + | | | | | |
| sulfuric acid | | | | | |
| 2.4% C581 | 6.44 | 9% | 2.1 | 2% | 13.2 |

### Example 19

### Foam Reduction in Bacillus subtilis Fermentation Broth Containing Surfactin

The reduction in foam formation in a Bacillus subtilis fermentation broth containing surfactin (described in Surfactin Clarified Solution section) was measured following sodium chloride treatment. Surfactin stock solution was prepared by adding 2.03 grams of de-ionized water directly to the vial containing surfactin and pH was adjusted between 6-7 (as measured by pH strip paper) using 1N NaOH. The stock solution was further diluted by adding 0.71 g of the stock solution to 1.9 g of B. subtilis fermentation broth prepared using techniques known in the art, and gently mixing the solution for 5 minutes. The surfactin containing broth was shaken 20 times and the appearance of the shaken sample was captured using digital camera. 0.022 g of NaCl was added to the same surfactin containing broth, shaken 20 times and photographed. An additional 0.046 g and 0.032 g of NaCl were added to the same broth sequentially and the broth shaken 20 times and photographed. Table 16 shows the total NaCl concentration in the broth after each treatment and the corresponding Overrun and Foam Reduction Index following each treatment.

TABLE 16
Treatment conditions and corresponding Overrun
and Foam Reduction Index in Bacillus subtilis
fermentation broth containing surfactin

| Treatment | Overrun | Foam Reduction Index |
|---|---|---|
| None | 22% | — |
| 0.8% NaCl | 3% | 6.7 |
| 2.5% NaCl | 1% | 15.9 |
| 3.7% NaCl | 0% | 83.9 |

### Example 20

### Foam Reduction in Bacillus licheniformis Fermentation Broth Containing Surfactin

The reduction in foam formation in a Bacillus licheniformis fermentation broth containing surfactin (described in Surfactin Clarified Solution section) was measured following calcium chloride treatment. Surfactin stock solution was prepared by adding 2.03 grams of de-ionized water directly to the vial containing surfactin and pH was adjusted between 6-7 (as measured by pH strip paper) using 1N NaOH. The stock solution was further diluted by adding 0.71 g of the stock solution to 1.9 g of B. licheniformis fermentation broth prepared using techniques known in the art, and gently mixing the solution for 5 minutes. The surfactin containing broth was shaken 20 times and the appearance of the shaken sample was captured using digital camera. 0.025 g of CaC₁₂ was added to the same surfactin containing broth, shaken 20 times and photographed. An additional 0.021 g CaCl₂ was added to the same containing broth, shaken 20 times and photographed. Table 17 shows the total calcium chloride concentration in the broth after each treatment and the corresponding Overrun and Foam Reduction Index following each treatment.

TABLE 17
Treatment conditions and corresponding Overrun
and Foam Reduction Index in Bacillus licheniformis
fermentation broth containing surfactin

| Treatment | Overrun | Foam Reduction Index |
|---|---|---|
| None | 65% | — |
| 1.0% calcium chloride | 27% | 2.4 |
| 1.9% calcium chloride | 10% | 6.4 |

FIG. 4 shows the appearance of the sample after each treatment described above.

The invention is further described by the following numbered paragraphs:

1. A method for controlling foaming of biosurfactant that foams during production thereof by a host cell in a fermentation medium when the host cell extracellularly secretes the biosurfactant and the biosurfactant is soluble in the fermentation medium, comprising, contemporaneously with production of the biosurfactant by the host cell, insolubilizing the biosurfactant, whereby foaming is controlled as the insolubilized biosurfactant does not foam.

2. The method of paragraph 1 wherein the biosurfactant comprises hydrophobin rhamnolipid, sophorolipid or surfactin.

3. The method of paragraph 2 wherein the insolubilizing comprises adding to the fermentation medium a precipitation agent and/or lowering pH of the fermentation medium and/or increasing temperature of the fermentation medium.

4. The method of paragraph 3 wherein the insolubilizing comprises adding to the fermentation medium a precipitation agent.

5. The method of paragraph 4 wherein the precipitation agent is a salt, alcohol, water miscible organic solvent, water soluble polymer or a cationic polymer.

6. The method of paragraph 5 wherein the precipitation agent is a salt that comprises as its anion a halide, a citrate, an acetate, a nitrate, a carbonate; a sulfate; a phosphate; a sulphamate; a phosphonate, a sulphamate, or is a nitrous acid salt and as its cation ammonium, calcium, iron, magnesium, lithium, potassium or sodium.

7. The method of paragraph 6 wherein the salt comprises a sulfate salt or a chloride salt.

8. The method of paragraph 7 wherein the chloride salt is calcium chloride or sodium chloride and the sulfate salt is ammonium sulfate or sodium sulfate.

9. The method of paragraph 5 wherein the precipitation agent is an alcohol and the alcohol comprises a monohydric or polyhydric alcohol C₁-C₆ alcohol.

10. The method of paragraph 5 where the precipitation agent solvent is a ketone.

11. The method of paragraph 10 where the ketone is acetone.

12. The method of paragraph 5 where the precipitation agent is polythyene glycol or a polysaccharide.

13. The method of paragraph 12 where the polysaccharide is dextran.

14. The method of paragraph 9 wherein the precipitation agent comprises methanol, ethanol or isopropyl alcohol.

15. The method of paragraph 3 wherein the insolubilizing comprises lowering pH of the fermentation medium and/or increasing temperature of the fermentation medium.

16. The method of any one of paragraphs 1-15 wherein the foam reduction index is greater than 1, and/or the foam reduction index is greater than 2, and/or the foam reduction index is greater than 3; and/or the concentration of soluble biosurfactant in the fermentation media is at most about 1 g/kg; and/or at least 25% of biosurfactant produced is insolubilized; and/or the method is performed without addition of antifoam; and/or the method is performed with a reduced amount of antifoam in comparison with the method run without insolubilizing; and/or the method is performed by raising or lowering the pH, and/or the method is performed by raising or lowering the temperature.

17. The method of any one of paragraphs 1-15 wherein the method is a continuous process comprising: feeding fermentation media to a bioreactor, adding precipitation agent or applying a precipitation condition, collecting insolubilized biosurfactant, and replenishing fermentation media or ingredients thereof or host cell; and optionally recycling any fermentation media or ingredients thereof or host cell collected with insolubilized biosurfactant.

18. The method of paragraph 16 wherein the method is a continuous process comprising: feeding fermentation media to a bioreactor, adding precipitation agent or applying a precipitation condition, collecting insolubilized biosurfactant, and replenishing fermentation media or ingredients thereof or host cell; and optionally recycling any fermentation media or ingredients thereof or host cell collected with insolubilized biosurfactant.

19. A method for controlling foaming of a biosurfactant that foams during production thereof by a host cell in a fermentation medium when the host cell extracellularly secretes the biosurfactant and the biosurfactant is soluble in the fermentation medium, comprising, contemporaneously with production of the biosurfactant by the host cell, insolubilizing the biosurfactant, whereby foaming is controlled as the insolubilized biosurfactant does not foam, wherein the foam reduction index is greater than 1, and/or the foam reduction index is greater than 2, and/or the foam reduction index is greater than 3; and/or the concentration of soluble biosurfactant in the fermentation media is at most about 1 g/kg; and/or at least 25% of the biosurfactant produced is insolubilized; and/or the method is performed without addition of antifoam; and/or the method is performed with a reduced amount of antifoam in comparison with the method run without insolubilizing the biosurfactant; and/or the method is performed by raising or lowering the pH, and/or the method is performed by raising or lowering the temperature.

20. The method of paragraph 19 wherein the biosurfactant comprises hydrophobin II, rhamnolipid, sophorolipid or surfactin.

21. The method of paragraph 19 or 20 wherein the insolubilizing comprises or consists essentially of adding to the fermentation medium a precipitation agent.

22. The method of paragraph 21 wherein the precipitation agent comprises or consists essentially of a salt that comprises as its anion a halide, a citrate, an acetate, a nitrate, a carbonate; a sulfate; a phosphate; a sulphamate; a phosphonate, a sulphamate, or is a nitrous acid salt and as its cation ammonium, calcium, iron, magnesium, lithium, potassium or sodium.

23. The method of paragraph 22 wherein the salt comprises or consists essentially of a sulfate.

24. The method of paragraph 21 wherein the precipitation agent comprises or consists essentially of an alcohol.

25. A method for controlling foaming of biosurfactant in a solution that foams during production, comprising:
contemporaneously during the production of the biosurfactant at points where conditions can give rise to foam formation, insolubilizing the biosurfactant, whereby foaming is controlled as the insolubilized biosurfactant does not foam.

26. The method of paragraph 25 wherein the solution comprises a fermentation medium, wherein the production comprises expression of the biosurfactant by a host cell in the fermentation medium, and wherein the host cell extracellularly secretes the biosurfactant and the biosurfactant is soluble in the fermentation medium whereby conditions can give rise to foam formation.

27. The method of paragraph 25 wherein the production comprises vacuum filtration whereby conditions can give rise to foam formation.

28. The method of paragraph 25 wherein the production comprises harvesting whereby conditions can give rise to foam formation.

29. The method of paragraph 25 wherein the production comprises collection whereby conditions can give rise to foam formation.

30. The method of paragraph 25 wherein the production comprises compaction whereby conditions can give rise to foam formation.

31. The method of paragraph 25 wherein the production comprises exsanguination whereby conditions can give rise to foam formation.

32. The method of paragraph 25 wherein the production comprises maceration whereby conditions can give rise to foam formation.

33. The method of paragraph 25 wherein the production comprises homogenization whereby conditions can give rise to foam formation.

34. The method of paragraph 25 wherein the production comprises mashing whereby conditions can give rise to foam formation.

35. The method of paragraph 25 wherein the production comprises brewing whereby conditions can give rise to foam formation.

36. The method of paragraph 25 wherein the production comprises recovery whereby conditions can give rise to foam formation.

37. The method of paragraph 25 wherein the production comprises solid-liquid separation whereby conditions can give rise to foam formation.

38. The method of paragraph 25 wherein the production comprises centrifugation whereby conditions can give rise to foam formation.

39. The method of paragraph 25 wherein the production comprises cell separation whereby conditions can give rise to foam formation.

40. The method of paragraph 25 wherein the production comprises any aerated process whereby conditions can give rise to foam formation.

41. The method of paragraph 25 wherein the production comprises pumping liquids, and/or filling equipment, and/or emptying equipment, and/or cleaning equipment, and/or rinsing equipment, whereby conditions can give rise to foam formation.

42. The method of paragraph 25 wherein the biosurfactant comprises hydrophobin II, rhamnolipid, sophorolipid or surfactin.

43. The method of paragraph 25 wherein insolubilizing the biosurfactant comprises:
adding a precipitation agent to the solution;
lowering or raising the pH of the solution; and/or
decreasing or increasing temperature of the solution.

44. The method of paragraph 25 wherein insolubilizing the biosurfactant comprises:

adding to the solution a precipitation agent.

45. The method of paragraph 43 or 44 wherein the precipitation agent is a salt, alcohol, water miscible organic solvent, or water soluble polymer or a cationic polymer.

46. The method of paragraph 43 or 44 wherein the precipitation agent is a salt that comprises as its anion a halide, a citrate, an acetate, a nitrate, a carbonate; a sulfate; a phosphate; a sulphamate; a phosphonate, a sulphamate, or is a nitrous acid salt and as its cation ammonium, calcium, iron, magnesium, lithium, potassium or sodium.

47. The method of paragraph 42 wherein the salt comprises a chloride salt or a sulfate salt.

48. The method of paragraph 43 wherein the chloride salt is calcium chloride or sodium chloride and the sulfate salt is ammonium sulfate or sodium sulfate.

49. The method of paragraph 42 wherein the precipitation agent is alcohol and the alcohol comprises a monhydric or polyhydric alcohol C₁-C₆ alcohol.

50. The method of paragraph 42 wherein the preciptiation agent comprises methanol, ethanol or isopropyl alcohol.

51. The method of paragraph 25 wherein insolubilizing the biosurfactant comprises:
lowering pH of the fermentation medium and/or
increasing temperature of the fermentation medium.

52. The method of any one of paragraphs 25-51 wherein the foam reduction index is greater than 1, and/or the foam reduction index is greater than 2, and/or the foam reduction index is greater than 3;
wherein the method is performed without addition of antifoam;
   wherein the method is performed with a reduced amount of antifoam in comparison with the method run without insolubilizing.

53. The method of any one of paragraphs 25-51 wherein the method is a continuous process comprising:
feeding fermentation media to a bioreactor;
adding precipitation agent or applying a precipitation condition;
collecting insolubilized biosurfactant; and
replenishing solution or ingredients thereof or host cell; and optionally recycling any solution or ingredients thereof or host cell collected with insolubilized biosurfactant.

54. The method of any one of paragraphs 25-51 wherein the concentration of soluble biosurfactant in the solution is less than about 10 g/kg.

55. The method of any one of paragraphs 25-51 wherein the concentration of soluble biosurfactant in the solution is in a range from about 0.1 g/kg to about 10 g/kg.

56. The method of any one of paragraphs 25-51 wherein the concentration of soluble biosurfactant in the solution is in a range from about 0.1 g/kg to about 5 g/kg.

57. The method of any one of paragraphs 25-51 wherein the concentration of soluble biosurfactant in the solution is in a range from about 0.1 g/kg to about 1.0 g/kg.

58. The method of any one of paragraphs 25-51 wherein at least 50% of biosurfactant produced is insolubilized.

59. The method of any one of paragraphs 25-51 wherein at least 75% of biosurfactant produced is insolubilized.

60. The method of any one of paragraphs 25-51 wherein at least 90% of biosurfactant produced is insolubilized.

61. The method of any one of paragraphs 25-51 wherein at least 95% of biosurfactant produced is insolubilized.

62. The method of any one of paragraphs 25-51 wherein the concentration of soluble biosurfactant in the solution is in a range from about 0.1 g/kg to about 10 g/kg and wherein at least 50% of biosurfactant produced is insolubilized.

63. The method of any one of paragraphs 25-51 wherein the solution comprises fermentation media.

64. A method for controlling foaming of biosurfactant that foams during production, comprising:
contemporaneously with production of the biosurfactant in a solution by the host cell, insolubilizing the biosurfactant,
controlling foaming such that:
   the foam reduction index is greater than 1, and/or the foam reduction index is greater than 2, and/or the foam reduction index is greater than 3; and/or
   the concentration of soluble biosurfactant in the solution is at most about 1 g/kg; and/or
   at least 25% of biosurfactant produced is insolubilized; and/or
   the method is performed without addition of antifoam; and/or
   the method is performed with a reduced amount of antifoam in comparison with the method run without insolubilizing.

65. The method of paragraph 64 wherein the solution is a fermentation medium;
wherein the production comprises expression of the biosurfactant by a host cell in the fermentation medium;
wherein the host cell extracellularly secretes the biosurfactant; and
wherein the biosurfactant is soluble in the fermentation medium whereby conditions can give rise to foam formation.

66. The method of paragraph 64 wherein the production comprises vacuum filtration whereby conditions can give rise to foam formation.

67. The method of paragraph 64 wherein the production comprises harvesting whereby conditions can give rise to foam formation.

68. The method of paragraph 64 wherein the production comprises collection whereby conditions can give rise to foam formation.

69. The method of paragraph 64 wherein the production comprises compaction whereby conditions can give rise to foam formation.

70. The method of paragraph 64 wherein the production comprises exsanguination whereby conditions can give rise to foam formation.

71. The method of paragraph 64 wherein the production comprises maceration whereby conditions can give rise to foam formation.

72. The method of paragraph 64 wherein the production comprises homogenization whereby conditions can give rise to foam formation.

73. The method of paragraph 64 wherein the production comprises mashing whereby conditions can give rise to foam formation.

74. The method of paragraph 64 wherein the production comprises brewing whereby conditions can give rise to foam formation.

75. The method of paragraph 64 wherein the production comprises recovery whereby conditions can give rise to foam formation.

76. The method of paragraph 64 wherein the production comprises solid liquid separation whereby conditions can give rise to foam formation.

77. The method of paragraph 64 wherein the production comprises centrifugation whereby conditions can give rise to foam formation.

78. The method of paragraph 64 wherein the production comprises cell separation whereby conditions can give rise to foam formation.

79. The method of paragraph 64 wherein the production comprises any aerated process whereby conditions can give rise to foam formation.

80. The method of paragraph 64 or 65 wherein the biosurfactant comprises hydrophobin II, rhamnolipid, sophorolipid or surfactin.

81. The method of paragraph 64, 64, or 80 wherein insolubilizing the biosurfactant comprises or consists essentially of adding to the solution a precipitation agent.

82. The method of paragraph 81 wherein the precipitation agent comprises or consists essentially of a salt that comprises as its anion a halide, a citrate, an acetate, a nitrate, a carbonate; a sulfate; a phosphate; a sulphamate; a phosphonate, a sulphamate, or is a nitrous acid salt and as its cation ammonium, calcium, iron, magnesium, lithium, potassium or sodium.

83. The method of paragraph 82 wherein the salt comprises or consists essentially of a sulfate.

84. The method of paragraph 81 wherein the precipitation agent comprises or consists essentially of an alcohol.

85. A method for controlling foaming of biosurfactant during production, comprising:
controlling conditions of a composition during production of the biosurfactant to reduce foam, comprising:
   adjusting conditions in the composition to reduce foaming such that:
      the foam reduction index is greater than 1, and/or the foam reduction index is greater than 2, and/or the foam reduction index is greater than 3;
      the concentration of soluble biosurfactant in the fermentation media is at most about 1 g/kg; and/or at least 25% of biosurfactant produced is insolubilized; and/or
      the method is performed without addition of antifoam; and/or the method is performed with a reduced amount of antifoam in comparison with the method run without insolubilizing.

86. The method of paragraph 85 wherein adjusting conditions in the composition comprises:
adjusting pH of the composition; and
adjusting a temperature of the composition.

87. The method of paragraph 85 comprising:
monitoring physical conditions of the composition during production to determine when foaming is occurring; and
providing a precipitating agent to the composition to reduce foaming.

88. The method of paragraph 87 wherein the precipitating agent comprises a salt, alcohol, water miscible organic solvent, water soluble polymer or a cationic polymer.

89. The method of paragraph 87 wherein the precipitation agent comprises a salt that comprises as its anion a halide, a citrate, an acetate, a nitrate, a carbonate; a sulfate; a phosphate; a sulphamate; a phosphonate, a sulphamate, or is a nitrous acid salt and as its cation ammonium, calcium, iron, magnesium, lithium, potassium or sodium.

90. The method of paragraph 89 wherein the salt comprises a chloride salt or a sulfate salt.

91. The method of paragraph 90 wherein the chloride salt is calcium chloride or sodium chloride and the sulfate salt is ammonium sulfate or sodium sulfate.

92. The method of paragraph 88 wherein the precipitation agent comprises an alcohol.

93. The method of any one of paragraphs 1, 2, 17-20, 26, 42, 53, 64, 65 or 80 wherein the host cell is Trichoderma reesei.

94. The method of any one of paragraphs 1, 2, 17-20, 26, 42, 53, 64, 65 or 80 wherein the host cell is Bacillus subtilis.

95. The method of any one of paragraphs 1, 2, 17-20, 26, 42, 53, 64, 65 or 80 wherein the host cell is Bacillus licheniformis.

96. The method of any one of paragraphs 1, 2, 17-20, 26, 42, 53, 64, 65 or 80 wherein the host cell is an Aspergillus species.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for controlling foaming of biosurfactant that foams during production thereof by a host cell in a fermentation medium when the host cell extracellularly secretes the biosurfactant and the biosurfactant is soluble in the fermentation medium, comprising, contemporaneously with production of the biosurfactant by the host cell, insolubilizing the biosurfactant, whereby foaming is controlled as the insolubilized biosurfactant does not foam.

2. The method of claim 1 wherein the biosurfactant comprises hydrophobin II, rhamnolipid, sophorolipid or surfactin.

3. The method of claim 1 wherein the foam reduction index is greater than 1, and/or the foam reduction index is greater than 2, and/or the foam reduction index is greater than 3; and/or the concentration of soluble biosurfactant in the fermentation media is at most about 1 g/kg; and/or at least 25% of biosurfactant produced is insolubilized; and/or the method is performed without addition of antifoam; and/or the method is performed with a reduced amount of antifoam in comparison with the method run without insolubilizing; and/or the method is performed by adding a precipitation agent and/or applying a precipitation condition.

4. The method of claim 1 wherein the method is a continuous process comprising: feeding fermentation media to a bioreactor, adding precipitation agent or applying a precipitation condition, collecting insolubilized biosurfactant, and replenishing fermentation media or ingredients thereof or host cell; and optionally recycling any fermentation media or ingredients thereof or host cell collected with insolubilized biosurfactant.

5. A method for controlling foaming of a biosurfactant that foams during production thereof by a host cell in a fermentation medium when the host cell extracellularly secretes the biosurfactant and the biosurfactant is soluble in the fermentation medium, comprising, contemporaneously with production of the biosurfactant by the host cell, insolubilizing the biosurfactant, whereby foaming is controlled as the insolubilized biosurfactant does not foam, wherein the foam reduction index is greater than 1, and/or the foam reduction index is greater than 2, and/or the foam reduction index is greater than 3; and/or the concentration of soluble biosurfactant in the fermentation media is at most about 1 g/kg; and/or at least 25% of the biosurfactant produced is insolubilized; and/or the method is performed without addition of antifoam; and/or the method is performed with a reduced amount of antifoam in comparison with the method run without insolubilizing the biosurfactant; and/or the method is performed by adding a precipitation agent and/or applying a precipitation condition.

6. A method for controlling foaming of biosurfactant in a solution that foams during production, comprising:
contemporaneously during the production of the biosurfactant at points where conditions can give rise to foam formation, insolubilizing the biosurfactant, whereby foaming is controlled as the insolubilized biosurfactant does not foam.

7. The method of claim 6 wherein the solution comprises a fermentation medium, wherein the production comprises expression of the biosurfactant by a host cell in the fermentation medium, and wherein the host cell extracellularly secretes the biosurfactant and the biosurfactant is soluble in the fermentation medium whereby conditions can give rise to foam formation.

8. The method of claim 6 wherein the method is a continuous process comprising:
feeding fermentation media to a bioreactor;
adding precipitation agent or applying a precipitation condition;
collecting insolubilized biosurfactant; and
replenishing solution or ingredients thereof or host cell; and optionally recycling any solution or ingredients thereof or host cell collected with insolubilized biosurfactant.

9. A method for controlling foaming of biosurfactant that foams during production, comprising:
contemporaneously with production of the biosurfactant in a solution by the host cell, insolubilizing the biosurfactant,
controlling foaming such that:
the foam reduction index is greater than 1, and/or the foam reduction index is greater than 2, and/or the foam reduction index is greater than 3; and/or
the concentration of soluble biosurfactant in the solution is at most about 1 g/kg; and/or
at least 25% of biosurfactant produced is insolubilized; and/or
the method is performed without addition of antifoam; and/or
the method is performed with a reduced amount of antifoam in comparison with the method run without insolubilizing.

10. The method of any one of claim 1, 5, 6 or 9 wherein the biosurfactant comprises hydrophobin II, rhamnolipid, sophorolipid or surfactin.

11. The method of any one of claim 1, 5, 6 or 9 wherein the host cell is Trichoderma reesei.

12. The method of any one of claim 1, 5, 6 or 9 wherein the host cell is Bacillus subtilis.

13. The method of any one of claim 1, 5, 6 or 9 wherein the host cell is Bacillus licheniformis.

14. The method of any one of claim 1, 5, 6 or 9 wherein the host cell is an Aspergillus species.
